(19) United States
(12) Patent Application Publication
(10) Pub. No.: US 2024/0327520 A1
(43) Pub. Date: Oct. 3, 2024

(54) METHODS AND COMPOSITIONS FOR PKC-DELTA INHIBITION AND CANCER IMMUNOTHERAPY

(71) Applicant: THE UNIVERSITY OF CHICAGO, Chicago, IL (US)

(72) Inventors: Thomas F. GAJEWSKI, Chicago, IL (US); Kyle R. CRON, Chicago, IL (US)

(73) Assignee: THE UNIVERSITY OF CHICAGO, Chicago, IL (US)

(21) Appl. No.: 18/708,530

(22) PCT Filed: Nov. 8, 2022
(86) PCT No.: PCT/US2022/079503

§ 371 Date: May 8, 2024

Related U.S. Application Data
(60) Provisional application No. 63/276,953, filed on Nov. 8, 2021.

(51) Int. Cl. C07K 16/28; A61K 31/352; A61K 31/403; A61K 38/17; A61K 39/00; A61K 39/395; A61P 35/00

(52) U.S. Cl. CPC C07K 16/2818; A61K 31/352; A61K 31/403; A61K 38/1709; A61K 39/3955; A61P 35/00; C07K 16/2827; A61K 2039/507

(57) ABSTRACT

Aspects of the present disclosure are directed to methods and compositions for PKC-delta inhibition and cancer immunotherapy. Disclosed are methods for treatment of cancer comprising administration of a PKC-delta inhibitor and an immunotherapy. Also disclosed are compositions comprising a PKC-delta inhibitor and an immunotherapeutic (e.g., an immune checkpoint inhibitor). Further described herein are methods for treating a subject for cancer using an immunotherapy, where the subject was determined to have reduced PKC-delta expression.

### CROSS-REFERENCE TO RELATED APPLICATIONS

This application claims the benefit of priority of U.S. Provisional Patent Application No. 63/276,953 filed Nov. 8, 2021, which is hereby incorporated by reference in its entirety.

### STATEMENT OF GOVERNMENT SUPPORT

This invention was made with government support under grant number CA210098, awarded by the National Institutes of Health. The government has certain rights in this invention.

### BACKGROUND

### I. Field of the Invention

Aspects of this invention relate to at least the fields of immunology, cancer biology, and medicine.

### II. Background

Checkpoint therapy is effective in tumors with already inflamed tumor microenvironments, however immunotherapy has limited effectiveness in patients with non-inflamed tumors. Thus, the ability to convert a non-inflamed into an inflamed tumor would open up previously unavailable therapies for a large number of cancer patients. However there are currently limited options for patients to accomplish this. There exists a need for compositions and methods capable of increasing an immune response to cancer and enhancing the efficacy of cancer immunotherapy.

### SUMMARY

Aspects of the present disclosure provide compositions and methods useful in increasing an anti-cancer immune response and enhancing efficacy of cancer immunotherapy such as immune checkpoint blockade therapy. As disclosed herein, reduced expression of PKC-delta in hematopoietic cells, including myeloid cells, increases anti-cancer immunity and improves response to checkpoint blockade therapy. Accordingly, disclosed herein are methods for treating a subject for cancer comprising administering a PKC-delta inhibitor and an immunotherapy. Also disclosed are therapeutic compositions comprising a PKC-delta inhibitor and immune checkpoint inhibitor (e.g., anti-PD1 antibody, anti-PD-L1 antibody, anti-CTLA4 antibody, etc.). Also contemplated herein is use of immunotherapy in the treatment of cancer subjects determined to have decreased PKC-delta expression relative to a control or reference subject.

Aspects of the present disclosure include methods for treating a subject for cancer, methods for evaluating PKC-delta expression in cells from a subject, methods for detecting a SNP in a subject, methods for targeting a PKC-delta inhibitor to myeloid cells, methods for targeting a PKC-delta inhibitor to myeloid cells, and pharmaceutical compositions. Methods of the disclosure can include at least 1, 2, 3, or more of the following steps: administering a PKC-delta inhibitor, administering an immunotherapy, administering a checkpoint blockade therapy, detecting a SNP, measuring PKC-delta expression levels, diagnosing a subject for cancer, targeting a PKC-delta inhibitor to a myeloid cell, targeting a PKC-delta inhibitor to a macrophage, subjecting immune cells to conditions sufficient to reduce PKC-delta expression, deleting PRKCD in a population of immune cells, and administering immune cells to a subject. Compositions of the disclosure can include at least 1, 2, 3, or more of the following components: a PKC-delta inhibitor, rotterlin, BJE6-106, delcasertib, a myeloid cell targeting agent, a macrophage targeting agent, an immune checkpoint inhibitor, an anti-PD1 antibody, an anti-PD-L1 antibody, and an anti-CTLA4 antibody.

Disclosed herein, in some aspects, is a method of treating a subject for cancer comprising administering to the subject a therapeutically effective amount of a PKC-delta inhibitor and an immunotherapy.

Also disclosed, in some aspects, is a method of treating a subject for cancer comprising administering to the subject a therapeutically effective amount of a PKC-delta inhibitor linked to a myeloid cell targeting agent. In some aspects, the method further comprises administering to the subject an immunotherapy.

Disclosed herein, in some aspects, is a method of treating a subject for cancer comprising administering an immunotherapy to a subject determined to have decreased PKC-delta expression relative to a control or reference subject. In some aspects, the subject was determined to have decreased PKC-delta expression in hematopoietic cells from the subject. In some aspects, the hematopoietic cells are myeloid cells. In some aspects, the subject was further determined to have a single nucleotide polymorphism (SNP) in the PRKCD gene. In some aspects, the SNP is rs1483185, rs1483186, or rs750170.

Further disclosed herein, in some aspects, is a method of treating a subject for cancer comprising administering an immunotherapy to a subject determined to have a SNP in the PRKCD gene, where the SNP is rs1483185, rs1483186, or rs750170. In some aspects, the subject was further determined to have decreased PKC-delta expression relative to a control or reference subject. In some aspects, the subject was determined to have decreased PKC-delta expression in hematopoietic cells from the subject. In some aspects, the hematopoietic cells are myeloid cells.

Disclosed herein, in some aspects, is a method of treating a subject for a RAS wild-type cancer comprising administering to the subject an effective amount of a PKC-delta inhibitor.

In some aspects, the immunotherapy is a checkpoint blockade therapy. In some aspects, the checkpoint blockade therapy comprises an anti-PD1 antibody, anti-PD-L1 antibody, or anti-CTLA4 antibody. In some aspects, the immunotherapy is an adoptive immune cell therapy. In some aspects, the immunotherapy is a CAR immune cell therapy. In some aspects, the PKC-delta inhibitor is a nucleic acid. In some aspects, the PKC-delta inhibitor is a small interfering RNA, a small hairpin RNA, or an antisense oligonucleotide. In some aspects, the PKC-delta inhibitor is rottlerin or an analog or derivative thereof. In some aspects, the PKC-delta inhibitor is BJE6-106. In some aspects, the PKC-delta inhibitor is delcasertib. In some aspects, the PKC-delta inhibitor is linked to a myeloid cell targeting agent. In some aspects, the myeloid cell targeting agent is an antibody that specifically binds to a myeloid cell surface protein. In some aspects, the myeloid cell surface protein is CD14, CD86, or CD16. In some aspects, the immunotherapy and the PKC-delta inhibitor are administered simultaneously. In some aspects, the immunotherapy and the PKC-delta inhibitor are administered sequentially. In some aspects, the immunotherapy is administered prior to administering the PKC-delta inhibitor. In some aspects, the immunotherapy is administered subsequent to administering the PKC-delta inhibitor.

Disclosed herein, in some aspects, is a method of treating a subject for cancer comprising: (a) subjecting immune cells ex vivo to conditions sufficient to reduce PKC-delta expression in the immune cells; and (b) subsequent to (a), administering the immune cells to the subject. In some aspects, (a) comprises deleting at least a portion of the PRKCD gene in the immune cells.

Disclosed herein, in some aspects, is a method of treating a subject for cancer comprising administering to the subject an effective amount of immune cells that do not express PKC-delta. In some aspects, the immune cells comprise a deletion in the PRKCD gene.

In some aspects, the immune cells comprise myeloid cells. In some aspects, the immune cells comprise CD8⁺ T cells. In some aspects, the method further comprises administering to the subject an additional immunotherapy. In some aspects, the additional immunotherapy is a checkpoint blockade therapy. In some aspects, the checkpoint blockade therapy comprises an anti-PD1 antibody, anti-PD-L1 antibody, or anti-CTLA4 antibody.

In some aspects, the subject has melanoma. In some aspects, the subject has lung cancer. In some aspects, the subject does not have a RAS mutant cancer. In some aspects, the subject was previously treated for cancer. In some aspects, the subject was determined to be resistant to the previous treatment.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

“Individual, “subject,” and “patient” are used interchangeably and can refer to a human or non-human.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-IC show a genome wide association study revealing a SNP located in the PRKCD gene associated with increased T cell gene expression and decreased PRKCD expression. (A) Manhattan plot of genome wide association results showing-log 10(p-values) for all SNPs ordered by chromosomal position. Blue dashed line indicates suggestive significance threshold (p-value <10⁻⁴), red dashed line indicates threshold for genome wide significance (p-value <10⁻⁷). Arrows point to SNPs located within the PRKCD gene. (B) T cell gene expression score for each patient, plotted by genotype at SNP rs1483185 (p-value=8.812e-08, Bonferroni corrected <0.05). (C) PKCδ expression in a lymphoblastoid cell line dataset, plotted by genotype at SNP rs1483185 (p-value=6e⁻⁰³).

FIGS. 2A-2J show hematopoietic loss of PKCδ leads to tumour outgrowth delay and altered immune response. (A) B16-SIY tumour volume growing in Prkcd^{+/+} bone marrow (BM) engrafted (black, N=5) or Prkcd^{-/-} BM engrafted animals (light grey, N=5). (B) B16-SIY tumour volume of animals engrafted with Prkcd^{+/+} BM (black) and treated with PBS vehicle (solid line, N=5), or anti-CD8p (dotted line, N=5) or engrafted with Prkcd^{-/-} BM (light grey) and treated with PBS vehicle (solid line, N=9), or anti-CD8β (dotted line, N=10). Treatments were administered 1 day before and 6 days after tumour injection. (C) Total number of splenic CD8⁺ T cells that stain positively for SIY tetramer by flow cytometery in Prkcd^{+/+} BM engrafted mice (black), or Prkcd^{-/-} BM engrafted mice (light grey) when mice are either tumour naive (left) or after 10 days of tumour growth (right). (D) Total number per gram of tumour of CD8⁺ T cells that stain positively for SIY tetramer by flow cytometry in Prkcd^{+/+} BM engrafted mice (black), or Prkcd^{-/-} BM engrafted mice (light grey) after 10 days of tumour growth. (E) The number of IFNg producing splenic cells after 10 days of tumour growth was determined by ELISPOT in Prkcd^{+/+} BM engrafted (black) and Prkcd^{-/-} BM engrafted mice (light grey) mice, with tumour naive Prkcd^{+/+} BM engrafted (dark grey) serving as a negative control. All groups were stimulated with either PBS vehicle (left) or SIY peptide (right). (F) Total number per gram of tumour of CD8⁺ T cells identified by flow cytometry in Prkcd^{-/-} BM engrafted mice (black), or Prkcd^{-/-} BM engrafted mice (light grey) after 10 days of tumour growth. (G) Total number per gram of tumour of FoxP3+T regulatory cells identified by flow cytometry in Prkcd^{+/+} BM engrafted mice (black), or Prkcd^{-/-} BM engrafted mice (light grey) after 10 days of tumour growth. (H) Total number per gram of tumour of CD8⁺ T cells identified by flow cytometry in Prkcd^{+/+} BM engrafted mice (black), or Prkcd^{-/-} BM engrafted mice (light grey) after 35 days of tumour growth. (I) Total number per gram of tumour of FoxP3⁺ T regulatory cells identified by flow cytometry in Prkcd^{+/+} BM engrafted mice (black), or Prkcd^{-/-} BM engrafted mice (light grey) after 35 days of tumour growth. (J) B16-SIY tumour volume of animals engrafted with Prkcd^{+/+} BM (black) and treated with PBS vehicle (solid line, N=7), or anti-PD-L1 (dotted line, N=6) or engrafted with Prkcd^{-/-} BM (light grey) and treated with PBS vehicle (solid line, N=7), or anti-PD-L1 (dotted line, N=7). Treatments are indicated with arrows and were administered 3×a week starting 26 days after tumour injection.

All data are mean +/− s.e.m., (A), (B), (G) significance determined by two-way ANOVA and Sidak's Multiple Comparisons test. **** [symbol] ≤0.0001. f significance determined by unpaired t test. *p [symbol] 0.05.

FIGS. 3A-3D show scRNAseq of CD45⁺ cells in the tumour microenvironment reveals a shift towards inflammation in the myeloid compartment of PKCδ KO animals. (A-D) CD45⁺dsRed-cells were sorted for scRNAseq from the tumours of Prkcd^{+/+} BM engrafted mice and Prkcd^{-/-} BM engrafted mice. (A) PKCδ expression data in Prkcd^{+/+} BM engrafted samples across all uniquely identified cell populations. (B) Number of differentially expressed genes between Prkcd^{-/-} BM engrafted and Prkcd^{-/-} BM engrafted populations within each cell population shown in a donut plot. Exact number of differentially expressed genes for each population is in legend. (C) Gene set enrichment analysis of differentially expressed genes identified in the C1qa⁺ myeloid cluster via scRNAseq with genes ranked from highest expression in PKCδ WT samples (left) to highest expression in PKCδ KO samples (right). Adjusted p value calculated using the fgsea R package. (D) Heatmap of log normalized expression of the top immune related differentially expressed genes in the C1qa⁺ myeloid cluster. Red gene names highlight genes of particular interest.

FIGS. 4A-4G show Loss of PKCδ in tumour associated macrophages alters inflammatory gene expression, synergizes with checkpoint therapy, and the gene expression profile predicts patient response to checkpoint therapy. (A) B16-SIY tumour volume in Prkcd^{fl/fl} Vavl-iCre⁻ (black, N=6) or Prkcd^{fl/fl} Vavl-iCre⁺ animals (grey, N=6). (B) RT-qPCR measuring expression of Trem2 and Illb relative to Gapdh in tumour associated macrophages (TAMs) sorted from Prkcd^{fl/fl} Vavl-iCre⁻ tumours (black) or Prkcd fl Vavl-iCre⁺ tumours (light grey) at day 24 endpoint. (C) Percentages of TAMs expressing CD14, CD69, and CD72 markers as measured by flow cytometry from Prkcd^{fl/fl} Vavl-iCre⁻ mice (black) or Prkcd^{fl/fl} Vavl-iCre⁺ mice (light grey) at day 24 endpoint. CD14:CD72 ratio was determined by taking the % of CD14⁺ macrophages divided by the % of CD72*macrophages. (D) B16-SIY tumour volume in Prkcd^{fl/fl} LysM-Cre⁻ (black, N=12) or Prkcd^{fl/fl} LysM-Cre⁺ animals (grey, N=11). (E) B16-SIY tumour volume in Prkcd^{fl/fl} LysM-Cre⁻ (black) animals treated with PBS (solid line, N=10), or anti-PD-L1 (dotted line, N=11), or Prkcd^{fl/fl} LysM-Cre⁺ (light grey) animals treated with PBS (solid line, N=8), or anti-PD-L1 (dotted line, N=9). Treatments are indicated with arrows and were administered 3× a week starting 7 days post tumour injection and ending after 5 doses. (F) Violin plot of patient M1/M2 gene signature scores from a TCGA skin cutaneous melanoma (SKCM) dataset, separated by homozygous minor, heterozygous, or homozygous major alleles at the rs1483185 SNP. Scores were compared between each group by ordinary one-way ANOVA and Tukey's multiple comparisons test. (G)

Violin plot of patient PKCd KO-like gene signature scores calculated from bulk RNAseq samples collected before anti-PD-L1 based therapy. Patients were then split by progression (progressive disease) or disease control (stable disease, partial response, complete response) and significance calculated by Student's T-test.

All data for a-d are mean +/− s.e.m., (A), (B), (F) significance determined by unpaired t test. * [symbol] 0.05, ** p≤ [symbol] 0.01, ***p≤ [symbol] 0.001, ****p [symbol] 0.0001 (C), (D) significance determined by two-way ANOVA and Sidak's Multiple Comparisons test. ***p≤ [symbol] 0.001.

FIGS. 5A-5E show SNP alleles correlate with PRKCD expression and the 160 gene signature score. (A) Expression of 82 genes in the immune gene signature score featured as a heatmap with 258 patients utilized in the GWAS. (B) Violin plot of patient 160 gene signature scores from a TCGA skin cutaneous melanoma (SKCM) dataset separated by homozygous minor, heterozygous, or homozygous major alleles at the SNP rs1483185. (C) Violin plot of patient 160 gene signature scores from a TCGA lung squamous cell carcinoma (LUSC) dataset separated by homozygous minor, heterozygous, or homozygous major alleles at the SNP rs1483185. (D) Violin plot of patient 160 gene signature scores from a TCGA skin cutaneous melanoma (SKCM) dataset separated by homozygous minor, heterozygous, or homozygous major alleles at the SNP rs1483186. (E) Violin plot of patient160 gene signature scores from a TCGA skin cutaneous melanoma (SKCM) dataset separated by homozygous minor, heterozygous, or homozygous major alleles at the SNP rs750170.

Scores for (C)-(F) were compared between each group by ordinary one-way ANOVA and Tukey's multiple comparisons test *p [symbol] 0.05, **p [symbol] 0.01, ***p [symbol] 0.001, ****p [symbol] 0.0001.

FIGS. 6A-6K show hematopoietic loss of PKCδ leads to an altered immune response. (A)-(J) bars on the left are Prkcd^{+/+} BM and bars on the right are Prkcd^{-/-} BM. (A) Percentages of splenic T cells populations per total CD3+ T cell population measured by flow cytometery in Prkcd^{+/+} BM engrafted mice (blue), or Prkcd^{-/-} BM engrafted mice (red) in naive mice. (B) Total number of splenic T cells populations measured by flow cytometery in Prkcd^{+/+} BM engrafted mice (blue), or Prkcd^{-/-} BM engrafted mice (red) in naive mice. (C) Percentage of splenic CD8⁺ T cells that stain positively for SIY tetramer by flow cytometry in Prkcd^{+/+} BM engrafted mice (blue), or Prkcd^{-/-} BM engrafted mice (red) when mice are either tumour naive (left) or after 10 days of tumour growth (right). (D) Percentages of splenic T cell populations per total CD3⁺ T cell population measured by flow cytometery in Prkcd^{+/+} BM engrafted mice (blue), or Prkcd^{-/-} BM engrafted mice (red) after 10 days of tumour growth. (E) Total number of splenic T cell populations measured by flow cytometery in Prkcd^{+/+} BM engrafted mice (blue), or Prkcd^{-/-} BM engrafted mice (red) after 10 days of tumour growth. (F) Total number per gram of tumour of CD3⁺ T cells identified by flow cytometry in Prkcd^{-/-} BM engrafted mice (blue), or Prkcd^{-/-} BM engrafted mice (red) after 35 days of tumour growth. (G) Total number per gram of tumour of conventional CD4⁺ T cells identified by flow cytometry in Prkcd^{+/+} BM engrafted mice (blue), or Prkcd^{-/-} BM engrafted mice (red) after 35 days of tumour growth. (H) Total number per gram of tumour of PD-1⁺, CD8⁺ T cells identified by flow cytometry in Prkcd^{+/+} BM engrafted mice (blue), or Prkcd^{-/-} BM engrafted mice (red) after 35 days of tumour growth. (I) Percentages of splenic T cell populations per total CD3+T cell population measured by flow cytometery in Prkcd^{-/-} BM engrafted mice (blue), or Prkcd^{-/-} BM engrafted mice (red) after 35 days of tumour growth. (J) Total number of splenic T cell populations measured by flow cytometery in Prkcd^{+/+} BM engrafted mice (blue), or Prkcd^{-/-} BM engrafted mice (red) after 35 days of tumour growth. (K) Survival curve of B16-SIY tumour bearing animals engrafted with Prkcd^{+/+} BM and treated with PBS vehicle (blue, N=7), or anti-PD-L1 (black, N=6) or engrafted with Prkcd^{-/-} BM and treated with PBS vehicle (red, N=7), or anti-PD-L1 (purple, N=7). Treatments were administered 3× a week starting 26 days after tumour injection.

All data for (A)-(J) are mean +/−s.e.m., (A)-(J)significance determined by unpaired t test. j significance determined by Kaplan-Meier survival analysis, p values for Gehan-Brewslow-Wilcoxon test reported. NS p>0.05*p [symbol] 0.05, **p≤ [symbol] 0.01, ***p≤ [symbol] 0.001, ****p≤ [symbol] 0.0001 FIG. 7 shows scRNAseq reveals distinct cell populations with many differentially expressed genes. UMAP projection of all cells collected, with all uniquely identified cell populations labeled by their cluster defining genes.

FIG. 8 shows an overview of the targeting strategy employed for creation of the Prkcd conditional knockout mice.

FIGS. 9A-9F show loss of PKCδ in tumour associated macrophages alters inflammatory gene expression and synergizes with checkpoint therapy in multiple Prkcd^{fl/fl} clones. (A) RT-qPCR measuring expression of Tnf relative to Gapdh in tumour associated macrophages (TAMs) sorted from Prkcd^{fl/fl} Vavl-iCre⁻ tumours (blue) or Prkcd^{fl/fl} Vavl-iCre⁺ tumours (red) at day 24 endpoint. (B) Percentages of TAMs expressing the marker CD86, as measured by flow cytometry from Prkcd^{fl/fl} Vavl-iCre⁻ mice (blue) or Prkcd^{fl/fl} Vavl-iCre*mice (red) at day 24 endpoint. (C) B16-SIY tumour volume in the alterative Prkcd^{fl/fl} clone “2C1” expressing either Vavl-iCre⁻ (blue, N=8) or Vavl-iCre⁺ (red, N=6). (D) Percentages of TAMs expressing CD14, CD69, CD72, and CD86 markers as measured by flow cytometry in alterative Prkcd^{fl/fl} clone “2C1” expressing either Vavl-iCre (blue) or Vavl-iCre⁺ mice (red) at day 24 endpoint. CD14:CD72 ratio was determined by taking the % of CD14+macrophages divided by the % of CD72*macrophages. (E) Survival curve of B16-SIY tumour bearing Prkcd^{fl/fl} LysM-Cre⁻ animals treated with PBS (blue, N=12), or anti-PD-L1 (black, N=11), or Prkcd^{fl/fl} LysM-Cre⁺ animals treated with PBS (red, N=11), or anti-PD-L1 (purple, N=11). Treatments were administered 3× a week starting 7 days post tumour injection and ending after 5 doses. (F) B16-SIY tumour volume in alterative Prkcd^{fl/fl} clone “2C1” expressing either LysM-Cre⁻ (blue) treated with PBS (solid line, N=13), or anti-PD-L1 (dotted line, N=14), or Prkcd^{fl/fl} LysM-Cre* (red) animals treated with PBS (solid line, N=11), or anti-PD-L1 (dotted line, N=11). Treatments are indicated with arrows and were administered 3×a week starting 7 days post tumour injection and ending after 5 doses.

All data for (A)-(D), (F) are mean +/− s.e.m., (A), (B), (D) significance determined by unpaired t test. * p> [symbol] 0.05, **p≤ [symbol] 0.01, ***p≤ [symbol] 0.001, **** p≤ [symbol] 0.0001 (C), (F) significance determined by two-way ANOVA and Sidak's Multiple Comparisons test. *** p≤ [symbol] 0.001, **** p [symbol] 0.0001. e significance determined by Kaplan-Meier survival analysis, p values for Gehan-Brewslow-Wilcoxon test reported. a***p [symbol] 0.001.

FIGS. 10A-10B show SNP alleles correlate with an M1/M2 gene signature score. (A) Violin plot of patient M1/M2 gene signature scores from a TCGA skin cutaneous melanoma (SKCM) dataset separated by homozygous minor, heterozygous, or homozygous major alleles at the SNP rs1483186. (B) Violin plot of patient M1/M2 gene signature scores from a TCGA skin cutaneous melanoma (SKCM) dataset separated by homozygous minor, heterozygous, or homozygous major alleles at the SNP rs750170.

Scores for (A), (B) were compared between each group by ordinary one-way ANOVA and Tukey's multiple comparisons test **p [symbol] 0.01, ***p [symbol] 0.001.

### DETAILED DESCRIPTION

Infiltration of tumors at baseline with CD8*T cells, along with expression of certain chemokine and interferon genes, are correlated with favorable clinical outcome to immunotherapy. As disclosed herein, an unbiased genome wide association study performed using The Cancer Genome Atlas (TCGA) data linked the expression of the gene PRKCD (PKC-delta or PKC-d) with expression of various T cell associated genes. The loss of PKC-d in the hematopoietic compartment caused a delay in tumor outgrowth which was dependent on CD8⁺ T cells. T cells were altered at the endpoint of these tumors and using single cell RNAseq it was determined this phenotype was accompanied by an alteration in the macrophage compartment. PKC-d KO animals had higher expression of M1 inflammatory genes and lower expression of M2 anti-inflammatory genes. PKC-d KO animals harboring tumors responded better to anti-PD-L1 therapy compared to WT animals.

Thus, the present disclosure is based, at least in part, on the surprising discovery that certain immune cells, including myeloid cells such as macrophages, that do not express PKC-delta (or have reduced PKC-delta expression) possess enhanced anti-tumor immunity. Accordingly, aspects of the present disclosure are directed to compositions and methods for cancer treatment involving inhibition of PKC-delta. Certain aspects are directed to methods for cancer therapy comprising treating a subject with a PKC-delta inhibitor and an immunotherapy (e.g., checkpoint blockade therapy). Additional aspects are directed to compositions comprising a PKC-delta inhibitor and an immune checkpoint inhibitor (e.g., anti-PD1 antibody, anti-PD-L1 antibody, anti-CTLA4 antibody, etc.). Further aspects are directed to adoptive cell therapy for cancer comprising treating a subject with immune cells that do not express PKC-delta.

Antibodies blocking the PD-1/PD-L1 axis and other immune checkpoints have revolutionized cancer care. Clinical response is favored in tumors showing a T cell-inflamed tumor microenvironment at baseline,^{1,2} which is tremendously variable between patients and also across tumor types.³ Despite the importance of this immunobiological phenotype, the mechanisms explaining such inter-patient heterogeneity are just beginning to be understood. Based on the notion that hypomorphic germline variants in immunoregulatory genes are linked to autoimmune diseases,⁴ one hypothesis is that germline variants might favor spontaneous immune priming and T cell infiltration into tumors. Certain aspects herein utilize TCGA data and identify germline variants in the PKCd gene associated with decreased expression of PRKCD and an increased immune gene signature in the tumor microenvironment. In some aspects, genetic deletion of PKCd in mice resulted in improved endogenous anti-tumor immunity and increased efficacy of PD-1/PD-L1 blockade. Single cell RNAseq of immune cells in the tumor revealed expression of Prkcd in myeloid cells, and PKCd deletion caused a macrophage shift from an M2 to an M1 phenotype. Conditional deletion of PKCd in macrophages recapitulated the improved tumor control phenotype and response to anti-PD-L1 treatment. Analysis of clinical samples from melanoma patients confirmed an association between PRKCD variants and M1/M2 phenotype, and between a PKCd KO-like gene signature and clinical benefit from anti-PD-1. In some aspects, reduced PKCd in host cells leads to improved anti-tumor immunity and PD-1 blockade efficacy through a myeloid shift to an M1 phenotype, and identify PKCd as a candidate therapeutic target.

### I. PKC-Delta and PKC-Delta Inhibitors

Aspects of the present disclosure include methods and compositions for inhibition of PKC-delta. PKC-delta (referred to elsewhere herein as “PKC-d” or “PKCδ” and also known as “protein kinase C delta type”) is a serine/threonine-protein kinase encoded by the PRKCD gene. An example mRNA sequence encoding for human PKC-delta is provided at NCBI RefSeq NM_001316327. An example protein sequence for human PKC-delta is provided at NCBI RefSeq NP_001303256.

Certain compositions and methods of the present disclosure comprise one or more PKC-delta inhibitors. As used herein, a “PKC-delta inhibitor” describes any molecule capable of 1) reducing expression of PKC-delta in a cell; and/or 2) inhibiting enzymatic activity of a PKC-delta protein. Accordingly, a PKC-delta inhibitor of the present disclosure may be, for example, an oligonucleotide or nucleic acid configured to reduce PKC-delta expression in a cell. Various types of inhibitory oligonucleotides and methods for generating such inhibitory oligonucleotides are recognized in the art, certain examples of which are described elsewhere herein.

A PKC-delta inhibitor of the present disclosure may be a small molecule PKC-delta inhibitor. PKC-delta inhibitors of the disclosure include molecules that preferentially inhibit PKC-delta over other protein kinase C molecules (e.g., PKC-alpha, PKC-beta, etc.). PKC-delta inhibitors of the disclosure also include molecules that inhibit PKC-delta with equivalent or reduced potency relative to other protein kinase C molecules. Various small molecule PKC-delta inhibitors are recognized in the art and contemplated herein and include, for example, PKC-delta inhibitors described in U.S. Pat. No. 9,364,460 and U.S. Patent Application Publication 2015/0283114, incorporated herein by reference in their entirety. Certain non-limiting examples of small molecule PKC-delta inhibitors which may be used in the disclosed compositions and methods include rotterlin (and analogs and derivatives thereof), BJE6-106, delcasertib, sotrastaurin, GSK690693, VTX-27, staurosporine, ruboxistaurin, and midostaurin (PKC412).

In some aspects, a PKC-delta inhibitor of the present disclosure is a compound of the formula:

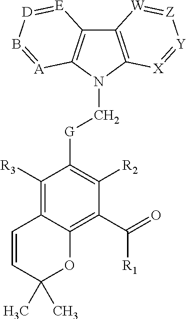

wherein:
R₁ is selected from the group consisting of H, methyl, ethyl, n-propyl, iso-propyl, n-butyl, sec-butyl, and tert-butyl;
R₂ and R₃ are each independently selected from the group consisting of H, OH and OR; A, B, D, E, W, X, Y, and Z are each independently selected from the group consisting of N and CH;
G is selected from the group consisting of O, NR, S, and CH₂,
R is selected from the group consisting of H, methyl, ethyl, n-propyl, iso-propyl, n-butyl, sec-butyl, tert-butyl, and aryl; and
n is an integer selected from the group consisting of 1, 2, 3, and 4.

In some cases, a PKC-delta inhibitor may be targeted to a particular cell or cell type to direct PKC-delta inhibition to the particular cell or cell type. In some aspects, a PKC-delta inhibitor is targeted to a cell that is not a cancer cell. For example, a PKC-delta inhibitor may be targeted to an immune cell. As disclosed herein, myeloid cells having reduced PKC-delta expression may possess enhanced anti-tumor immunity. Accordingly, in some aspects, aspects of the disclosure are directed to methods for targeting a PKC-delta inhibitor to a myeloid cell in a subject, thereby treating the subject for cancer. In some aspects, a PKC-delta inhibitor is linked (e.g., covalently or noncovalently linked) to a myeloid cell targeting agent. As used herein, a “myeloid cell targeting agent” describes any molecule capable of preferentially binding to a surface of a myeloid cell. In some aspects, a myeloid cell targeting agent of the disclosure is an antibody (or antibody fragment or antibody-like molecule) capable of preferentially binding to a protein on a surface of a myeloid cell (a “myeloid cell surface protein”). In some aspects, a myeloid cell targeting agent of the disclosure is a ligand for a myeloid cell surface protein. Various cell surface proteins expressed by myeloid cells are recognized in the art and contemplated herein.

In some aspects, a myeloid cell surface protein is CD14. Accordingly, aspects of the present disclosure include a composition comprising a PKC-delta inhibitor linked to an anti-CD14 antibody. Additional aspects are directed to a composition comprising a PKC-delta inhibitor linked to a CD14 binding molecule (e.g., lipopolysaccharide). Further aspects include as methods of administration of such compositions to a subject for treatment of cancer.

In some aspects, a myeloid cell surface protein is CD69. Accordingly, aspects of the present disclosure include a composition comprising a PKC-delta inhibitor linked to an anti-CD69 antibody. Additional aspects are directed to a composition comprising a PKC-delta inhibitor linked to a CD69 binding molecule. Further aspects include as methods of administration of such compositions to a subject for treatment of cancer.

In some aspects, a myeloid cell surface protein is CD86. Accordingly, aspects of the present disclosure include a composition comprising a PKC-delta inhibitor linked to an anti-CD86 antibody. Additional aspects are directed to a composition comprising a PKC-delta inhibitor linked to a CD86 binding molecule (e.g., CTLA-4 or portion thereof). Further aspects include as methods of administration of such compositions to a subject for treatment of cancer.

In some aspects, a myeloid cell surface protein is CD16. Accordingly, aspects of the present disclosure include a composition comprising a PKC-delta inhibitor linked to an anti-CD16 antibody. Additional aspects are directed to a composition comprising a PKC-delta inhibitor linked to a CD16 binding molecule (e.g., an IgG molecule or portion thereof). Further aspects include as methods of administration of such compositions to a subject for treatment of cancer.

### II. Cancer Therapy

In some aspects, the disclosed methods comprise administering a cancer therapy to a subject or patient. The cancer therapy may be chosen based on an expression level measurement, alone or in combination with a clinical risk score calculated for the subject. The cancer therapy may be chosen based on a genotype of a subject. The cancer therapy may be chosen based on the presence or absence of one or more polymorphisms in a subject. In some aspects, the cancer therapy comprises a local cancer therapy. In some aspects, the cancer therapy excludes a systemic cancer therapy. In some aspects, the cancer therapy excludes a local therapy. In some aspects, the cancer therapy comprises a local cancer therapy without the administration of a system cancer therapy. In some aspects, the cancer therapy comprises an immunotherapy, which may be a checkpoint inhibitor therapy. Any of these cancer therapies may also be excluded. Combinations of these therapies may also be administered.

The term “cancer,” as used herein, may be used to describe a solid tumor, metastatic cancer, or non-metastatic cancer. In certain aspects, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some aspects, the cancer is a Stage I cancer. In some aspects, the cancer is a Stage II cancer. In some aspects, the cancer is a Stage III cancer. In some aspects, the cancer is a Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some aspects, the cancer is melanoma. In some aspects, the cancer is lung cancer. In some aspects, the cancer is a RAS mutant cancer (i.e. a cancer having a RAS mutation). In some aspects, the cancer is not a RAS mutant cancer.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

### A. Radiotherapy

In some aspects, a radiotherapy, such as ionizing radiation, is administered to a subject. As used herein, “ionizing radiation” means radiation comprising particles or photons that have sufficient energy or can produce sufficient energy via nuclear interactions to produce ionization (gain or loss of electrons). A non-limiting example of ionizing radiation is x-radiation. Means for delivering x-radiation to a target tissue or cell are well known in the art.

In some aspects, the radiotherapy can comprise external radiotherapy, internal radiotherapy, radioimmunotherapy, or intraoperative radiation therapy (IORT). In some aspects, the external radiotherapy comprises three-dimensional conformal radiation therapy (3D-CRT), intensity modulated radiation therapy (IMRT), proton beam therapy, image-guided radiation therapy (IGRT), or stereotactic radiation therapy. In some aspects, the internal radiotherapy comprises interstitial brachytherapy, intracavitary brachytherapy, or intraluminal radiation therapy. In some aspects, the radiotherapy is administered to a primary tumor.

In some aspects, the amount of ionizing radiation is greater than 20 Gy and is administered in one dose. In some aspects, the amount of ionizing radiation is 18 Gy and is administered in three doses. In some aspects, the amount of ionizing radiation is at least, at most, or exactly 0.5, 1, 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 Gy (or any derivable range therein). In some aspects, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some aspects, the amount of radiotherapy administered to a subject may be presented as a total dose of radiotherapy, which is then administered in fractionated doses. For example, in some aspects, the total dose is 50 Gy administered in 10 fractionated doses of 5 Gy each. In some aspects, the total dose is 50-90 Gy, administered in 20-60 fractionated doses of 2-3 Gy each. In some aspects, the total dose of radiation is at least, at most, or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 Gy (or any derivable range therein). In some aspects, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein). In some aspects, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some aspects, at least, at most, or exactly 1, 2, 3, 4, 5, 6, 7, 8, 9, 10, 11, or 12 (or any derivable range therein) fractionated doses are administered per day. In some aspects, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### B. Cancer Immunotherapy

In some aspects, the methods comprise administration of a cancer immunotherapy. Cancer immunotherapy (sometimes called immuno-oncology, abbreviated IO) is the use of the immune system to treat cancer. Immunotherapies can, in some cases, be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and certain examples are described below.

### 1. Checkpoint Inhibitors and Combination Treatment

Aspects of the disclosure may include administration of immune checkpoint inhibitors, examples of which are further described below. As disclosed herein, “checkpoint inhibitor therapy” (also “immune checkpoint blockade therapy,” “checkpoint blockade therapy,” “immune checkpoint therapy,” “ICT,” “checkpoint blockade immunotherapy,” or “CBI”), refers to cancer therapy comprising providing one or more immune checkpoint inhibitors to a subject suffering from or suspected of having cancer.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some aspects, PD-1, PDL1, and PDL2 are human PD-1, PDL1 and PDL2.

In some aspects, the PD-1 inhibitor is a molecule that inhibits the binding of PD-1 to its ligand binding partners. In a specific aspect, the PD-1 ligand binding partners are PDL1 and/or PDL2. In another aspect, a PDL1 inhibitor is a molecule that inhibits the binding of PDL1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-I inhibitor is an anti-PD-I antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-I inhibitor is an immunoadhesin (e.g., an immunoadhesin comprising an extracellular or PD-1 binding portion of PDL1 or PDL2 fused to a constant region (e.g., an Fc region of an immunoglobulin sequence). In some aspects, the PDL1 inhibitor comprises AMP-224. Nivolumab, also known as MDX-1106-04, MDX-1106, ONO-4538, BMS-936558, and OPDIVO®, is an anti-PD-I antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-I inhibitors include MEDI0680, also known as AMP-514, and REGN2810.

In some aspects, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the V_{H} region of nivolumab, pembrolizumab, or pidilizumab, and the CDR1, CDR2 and CDR3 domains of the V_{L} region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4 or CTLA4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or V_{H} and/or V_{L} domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the V_{H} region of tremelimumab or ipilimumab, and the CDR1, CDR2 and CDR3 domains of the V_{L} region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

c. LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8′ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some aspects, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or V_{H} and/or V_{L} domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, B1754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (I1MP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4(Supp. 1):P195 (REGN3767); Yu, X., et al., mAbs, 2019; 11:6 (LBL-007) can be used in the methods disclosed herein. These and other anti-LAG-3 antibodies useful in the claimed invention can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some aspects, the inhibitor comprises the heavy and light chain CDRs or VRs of an anti-LAG3 antibody. Accordingly, in one aspect, the inhibitor comprises the CDR1, CDR2, and CDR3 domains of the V_{H} region of an anti-LAG3 antibody, and the CDR1, CDR2 and CDR3 domains of the V_{L} region of an anti-LAG3 antibody. In another aspect, the antibody has at least about 70, 75, 80, 85, 90, 95, 97, or 99% (or any derivable range therein) variable region amino acid sequence identity with the above-mentioned antibodies. d. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM 032782. TIM-3 is found on the surface IFNγ-producing CD4⁺ Th1 and CD8⁺ Tcl cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4⁺ Thl-specific cell surface protein that regulates macrophage activation. Inhibitors of the disclosure may block one or more functions of TIM-3 activity.

In some aspects, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or V_{H} and/or V_{L} domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed invention can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to TIM-3 also can be used.

In some aspects, the inhibitor comprises the heavy and light chain CDRs or VRs of an anti-TIM-3 antibody. Accordingly, in one aspect, the inhibitor comprises the CDR1, CDR2, and CDR3 domains of the V_{H} region of an anti-TIM-3 antibody, and the CDR1, CDR2 and CDR3 domains of the V_{L} region of an anti-TIM-3 antibody. In another aspect, the antibody has at least about 70, 75, 80, 85, 90, 95, 97, or 99% (or any derivable range or value therein) variable region amino acid sequence identity with the above-mentioned antibodies.

2. Activator of co-stimulatory molecules

In some aspects, the immunotherapy comprises an activator (also “agonist”) of a co-stimulatory molecule. In some aspects, the agonist comprises an agonist of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Agonists include activating antibodies, polypeptides, compounds, and nucleic acids.

3. Dendritic cell therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

4. CAR-T cell therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell, natural killer (NK) cell, or other immune cell. The receptors are called chimeric because they are fused of parts from different sources. CAR-T cell therapy refers to a treatment that uses such transformed cells for cancer therapy, where the transformed cells are T cells. Similar therapies include, for example, CAR-NK cell therapy, which uses transformed NK cells.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Example CAR-T therapies include Tisagenlecleucel (Kymriah) and Axicabtagene ciloleucel (Yescarta).

### 5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an example interleukin cytokine therapy.

### 6. Adoptive Cell Therapy

Adoptive cell therapy is a form of passive immunization by the transfusion of immune cells, such as T cells, NK cells, or other immune cells (also called “adoptive cell transfer”). Immun cells used for adoptive cell therapy include those found in normal tissue and those found in tumor tissue (where they are known as tumor infiltrating immune cells or tumor infiltrating lymphocytes). Although tumor infiltrating immune cells can attack a tumor, the environment within the tumor is generally highly immunosuppressive, preventing immune-mediated tumor death.

Multiple ways of producing and obtaining tumor targeted immune cells have been developed. Immune cells specific to a tumor antigen can be removed from a tumor sample or filtered from blood. Subsequent activation and culturing may be performed ex vivo, with the results reinfused. Activation can take place through gene therapy, by exposing the immune cells to tumor antigens, or by other methods known in the art.

As disclosed herein, immune cells having reduced or no expression of PRKCD may have enhanced anti-tumor immunity. Accordingly, in certain aspects, an adoptive cell therapy of the disclosure comprises administration of immune cells that do not express, or have reduced expression of, PRKCD. For example, an adoptive cell therapy of the disclosure may comprise obtaining immune cells from a subject, eliminating PRKCD gene expression in the cells (e.g., by generating a deletion or other mutation in the PRCKD gene), and administering the cells to the same subject or a different subject.

### 7. Oncolytic Virus

In some aspects, the cancer therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy.

### C. Chemotherapies

In some aspects, a therapy of the present disclosure comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some aspects, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection.

Other suitable chemotherapeutic agents include antimicrotubule agents, e.g., Paclitaxel (“Taxol”) and doxorubicin hydrochloride (“doxorubicin”). Doxorubicin is absorbed poorly and is preferably administered intravenously. In certain aspects, appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred in certain cases. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

The amount of the chemotherapeutic agent delivered to a patient may be variable. In one suitable aspect, the chemotherapeutic agent may be administered in an amount effective to cause arrest or regression of the cancer in a host, when the chemotherapy is administered with the construct. In other aspects, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### D. Hormone Therapy

In some aspects, a cancer therapy of the present disclosure is a hormone therapy. In particular aspects, a prostate cancer therapy comprises hormone therapy. Various hormone therapies are known in the art and contemplated herein. Examples of hormone therapies include, but are not limited to, luteinizing hormone-releasing hormone (LHRH) analogs, LHRH antagonists, androgen receptor antagonists, and androgen synthesis inhibitors.

### E. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### F. Additional Cancer Therapies

Therapeutic methods disclosed herein may comprise one or more additional cancer therapies. A cancer therapy of the disclosure may comprise, for example, cryoablative therapy, high-intensity ultrasound (also “high-intensity focused ultrasound”), photodynamic therapy, laser ablation, and/or irreversible electroporation. A cancer therapy of the disclosure may comprise 1, 2, 3, 4, 5, or more distinct therapeutic methods.

It is contemplated that a cancer treatment may exclude any of the cancer treatments described herein. Furthermore, aspects of the disclosure include patients that have been previously treated for a therapy described herein, are currently being treated for a therapy described herein, or have not been treated for a therapy described herein. In some aspects, the patient is one that has been determined to be resistant to a therapy described herein. In some aspects, the patient is one that has been determined to be sensitive to a therapy described herein.

### III. Administration of Therapeutic Combinations

The therapy provided herein may comprise administration of a combination of therapeutic agents, such as a PKC-delta inhibitor and an immunotherapy (e.g., an immune checkpoint blockade therapy). The therapies may be administered in any suitable manner known in the art. For example, the PKC-delta inhibitor and the immunotherapy may be administered sequentially (at different times) or concurrently (at the same time). In some embodiments, the PKC-delta inhibitor and the immunotherapy are administered in a separate composition. In some embodiments, the PKC-delta inhibitor and the immunotherapy are in the same composition.

In some embodiments, the PKC-delta inhibitor and the immunotherapy are administered substantially simultaneously. In some embodiments, the PKC-delta inhibitor and the immunotherapy are administered sequentially. In some embodiments, the PKC-delta inhibitor, the immunotherapy, and an additional cancer therapy (e.g., chemotherapy) are administered sequentially. In some embodiments, the PKC-delta inhibitor is administered before administering the immunotherapy. In some embodiments, the PKC-delta inhibitor is administered after administering the immunotherapy.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. For example, one therapeutic agent (e.g., a PKC-delta inhibitor) may be administered by one route of administration (e.g., orally) while a second therapeutic agent (e.g., an immune checkpoint inhibitor) may be administered by a different route of administration (e.g., intravenously). In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

### IV. Inhibitory Oligonucleotides

In some aspects, the disclosure relates to inhibitory oligonucleotides that inhibit the gene expression of PRKCD. Examples of inhibitory oligonucleotides include but are not limited to small interfering RNA (siRNA), short hairpin RNA (shRNA), double-stranded RNA, an antisense oligonucleotide, a ribozyme, and an oligonucleotide encoding any thereof. An inhibitory oligonucleotide may inhibit the transcription of a gene or prevent the translation of a gene transcript in a cell. An inhibitory oligonucleotide acid may be from 16 to 1000 nucleotides long, and in certain embodiments from 18 to 100 nucleotides long. The oligonucleotide may have at least or may have at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, or 90 (or any range derivable therein) nucleotides. The oligonucleotide may be DNA, RNA, or a cDNA that encodes an inhibitory RNA.

As used herein, “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered.

Inhibitory oligonucleotides are well known in the art. For example, siRNA and double-stranded RNA have been described in U.S. Pat. Nos. 6,506,559 and 6,573,099, as well as in U.S. Patent Publications 2003/0051263, 2003/0055020, 2004/0265839, 2002/0168707, 2003/0159161, and 2004/0064842, all of which are herein incorporated by reference in their entirety.

In some aspects, an inhibitory oligonucleotide may be capable of decreasing the expression of PRKCD by at least, at most, or about 10%, 20%, 30%, 40%, 50%, 60%, or 70%, 75%, 80%, 90%, 95%, 99%, or 100%, or any range or value in between the foregoing.

In further embodiments, disclosed are synthetic oligonucleotides that are PKC-delta inhibitors (e.g., synthetic oligonucleotides configured to reduce expression of PRKCD). An inhibitor may be between 17 to 25 nucleotides in length and comprise a 5′ to 3′ sequence that is at least 90% complementary to the 5′ to 3′ sequence of a mature PRKCD mRNA. In certain embodiments, an inhibitor molecule is 17, 18, 19, 20, 21, 22, 23, 24, or 25 nucleotides in length, or any range derivable therein. In some aspects, an inhibitor molecule has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a region of a mature PRKCD mRNA, particularly a mature, naturally occurring PRKCD mRNA. One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

In some embodiments, the inhibitory oligonucleotide is an analog and may include modifications, particularly modifications that increase nuclease resistance, improve binding affinity, and/or improve binding specificity. For example, when the sugar portion of a nucleoside or nucleotide is replaced by a carbocyclic moiety, it is no longer a sugar. Moreover, when other substitutions, such a substitution for the inter-sugar phosphodiester linkage are made, the resulting material is no longer a true species. All such compounds are considered to be analogs. Throughout this specification, reference to the sugar portion of a nucleic acid species shall be understood to refer to either a true sugar or to a species taking the structural place of the sugar of wild type nucleic acids. Moreover, reference to inter-sugar linkages shall be taken to include moieties serving to join the sugar or sugar analog portions in the fashion of wild type nucleic acids.

Aspects of the present disclosure concern modified oligonucleotides, i.e., oligonucleotide analogs or oligonucleosides, and methods for effecting the modifications. These modified oligonucleotides and oligonucleotide analogs may exhibit increased chemical and/or enzymatic stability relative to their naturally occurring counterparts. Extracellular and intracellular nucleases generally do not recognize and therefore do not bind to the backbone-modified compounds. When present as the protonated acid form, the lack of a negatively charged backbone may facilitate cellular penetration.

The modified internucleoside linkages are intended to replace naturally-occurring phosphodiester-5′-methylene linkages with four atom linking groups to confer nuclease resistance and enhanced cellular uptake to the resulting compound.

Modifications may be achieved using solid supports which may be manually manipulated or used in conjunction with a DNA synthesizer using methodology commonly known to those skilled in DNA synthesizer art. Generally, the procedure involves functionalizing the sugar moieties of two nucleosides which will be adjacent to one another in the selected sequence. In a 5′ to 3′ sense, an “upstream” synthon such as structure H is modified at its terminal 3′ site, while a “downstream” synthon such as structure H1 is modified at its terminal 5′ site.

Oligonucleosides linked by hydrazines, hydroxylarnines, and other linking groups can be protected by a dimethoxytrityl group at the 5′-hydroxyl and activated for coupling at the 3′-hydroxyl with cyanoethyldiisopropyl-phosphite moieties. These compounds can be inserted into any desired sequence by standard, solid phase, automated DNA synthesis techniques. One of the most popular processes is the phosphoramidite technique. Oligonucleotides containing a uniform backbone linkage can be synthesized by use of CPG-solid support and standard nucleic acid synthesizing machines such as Applied Biosystems Inc. 380B and 394 and Milligen/Biosearch 7500 and 8800s. The initial nucleotide (number 1 at the 3′-terminus) is attached to a solid support such as controlled pore glass. In sequence specific order, each new nucleotide is attached either by manual manipulation or by the automated synthesizer system.

Free amino groups can be alkylated with, for example, acetone and sodium cyanoboro hydride in acetic acid. The alkylation step can be used to introduce other, useful, functional molecules on the macromolecule. Such useful functional molecules include but are not limited to reporter molecules, RNA cleaving groups, groups for improving the pharmacokinetic properties of an oligonucleotide, and groups for improving the pharmacodynamic properties of an oligonucleotide. Such molecules can be attached to or conjugated to the macromolecule via attachment to the nitrogen atom in the backbone linkage. Alternatively, such molecules can be attached to pendent groups extending from a hydroxyl group of the sugar moiety of one or more of the nucleotides. Examples of such other useful functional groups are provided by WO1993007883, which is herein incorporated by reference, and in other of the above-referenced patent applications.

Solid supports may include any of those known in the art for polynucleotide synthesis, including controlled pore glass (CPG), oxalyl controlled pore glass, TentaGel Support—an aminopolyethyleneglycol derivatized support or Poros—a copolymer of polystyrene/divinylbenzene. Attachment and cleavage of nucleotides and oligonucleotides can be effected via standard procedures. As used herein, the term solid support further includes any linkers (e.g., long chain alkyl amines and succinyl residues) used to bind a growing oligonucleoside to a stationary phase such as CPG. In some embodiments, the oligonucleotide may be further defined as having one or more locked nucleotides, ethylene bridged nucleotides, peptide nucleic acids, or a 5′(E)-vinyl-phosphonate (VP) modification. In some embodiments, the oligonucleotides has one or more phosphorothioated DNA or RNA bases.

### V. Other Agents

It is contemplated that other agents may be used in combination with certain aspects of the present embodiments to improve the therapeutic efficacy of treatment. These additional agents include agents that reduce expression of PRKCD and/or reduce activity of PKC-delta.

### VI. Pharmaceutical Compositions

In some embodiments, pharmaceutical compositions are administered to a subject. Different aspects may involve administering an effective amount of a composition to a subject. In some embodiments, a composition comprises a PKC-delta inhibitor. Additionally, such compositions can be administered in combination with an additional therapeutic agent (e.g., a chemotherapeutic, an immunotherapeutic, a biotherapeutic, etc.). Such compositions will generally be dissolved or dispersed in a pharmaceutically acceptable carrier or aqueous medium.

The present disclosure includes methods for treating disease and modulating immune responses in a subject in need thereof. The disclosure includes cells that may be in the form of a pharmaceutical composition that can be used to induce or modify an immune response.

Administration of the compositions according to the current disclosure will typically be via any common route. This includes, but is not limited to parenteral, orthotopic, intradermal, subcutaneous, orally, transdermally, intramuscular, intraperitoneal, intraperitoneally, intraorbitally, by implantation, by inhalation, intraventricularly, intranasally or intravenous injection. In some embodiments, compositions of the present disclosure (e.g., compositions comprising GHR-binding polypeptides) are administered to a subject intravenously.

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most or at least 3, 4, 5, 6, 7, 8, 9, 10 or more. The administrations may range from 2-day to 12-week intervals, more usually from one to two week intervals.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

Pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (e.g., polypeptides of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

The compositions and related methods of the present disclosure, particularly administration of a composition of the disclosure may also be used in combination with the administration of additional therapies such as the additional therapeutics described herein or in combination with other traditional therapeutics known in the art.

The therapeutic compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 pg/kg, mg/kg, pg/day, or mg/day or any range derivable therein. Furthermore, such doses can be administered at multiple times during a day, and/or on multiple days, weeks, or months.

In some embodiments, the therapeutically effective or sufficient amount of the immune checkpoint inhibitor, such as an antibody and/or microbial modulator, that is administered to a human will be in the range of about 0.01 to about 50 mg/kg of patient body weight whether by one or more administrations. In some embodiments, the therapy used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one embodiment, a therapy described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VII. Cell Culture

In some embodiments, cells may be cultured for at least between about 10 days and about 40 days, for at least between about 15 days and about 35 days, for at least between about 15 days and 21 days, such as for at least about 15, 16, 17, 18, 19 or 21 days. In some embodiments, the cells of the disclosure may be cultured for no longer than 60 days, or no longer than 50 days, or no longer than 45 days. The cells may be cultured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days. The cells may be cultured in the presence of a liquid culture medium. Typically, the medium may comprise a basal medium formulation as known in the art. Many basal media formulations can be used to culture cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art, and it is within the skill of one in the art to modify or modulate concentrations of media and/or media supplements as necessary for the cells cultured. In some embodiments, a culture medium formulation may be explants medium (CEM) which is composed of IMDM supplemented with 10% fetal bovine serum (FBS), 100 U/ml penicillin G, 100 μg/ml streptomycin and 2 mmol/L L-glutamine. Other embodiments may employ further basal media formulations, such as chosen from the ones above.

Any medium capable of supporting cells in vitro may be used to culture the cells. Media formulations that can support the growth of cells include, but are not limited to, Dulbecco's Modified Eagle's Medium (DMEM), alpha modified Minimal Essential Medium (αMEM), and Roswell Park Memorial Institute Media 1640 (RPMI Media 1640) and the like. Typically, up to 20% fetal bovine serum (FBS) or 1-20% horse serum is added to the above medium in order to support the growth of cells. A defined medium, however, also can be used if the growth factors, cytokines, and hormones necessary for culturing cells are provided at appropriate concentrations in the medium. Media useful in the methods of the disclosure may comprise one or more compounds of interest, including, but not limited to, antibiotics, mitogenic compounds, or differentiation compounds useful for the culturing of cells. The cells may be grown at temperatures between 27° C. to 40° C., such as 31° C. to 37° C., and may be in a humidified incubator. The carbon dioxide content may be maintained between 2% to 10% and the oxygen content may be maintained between 1% and 22%. The disclosure, however, should in no way be construed to be limited to any one method of isolating and culturing cells. Rather, any method of isolating and culturing cells should be construed to be included in the present disclosure.

For use in the cell culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., P-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. Also contemplated is supplementation of cell culture medium with mammalian plasma or sera. Plasma or sera often contain cellular factors and components that are necessary for viability and expansion. The use of suitable serum replacements is also contemplated.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed. In particular embodiments, cells are cultured in a cell culture system comprising a cell culture medium, preferably in a culture vessel, in particular a cell culture medium supplemented with a substance suitable and determined for protecting the cells from in vitro aging and/or inducing in an unspecific or specific reprogramming.

### VIII. Cell Generation

Certain methods of the disclosure concern culturing the cells obtained from human tissue samples. In particular embodiments of the present disclosure, cells are plated onto a substrate that allows for adherence of cells thereto. This may be carried out, for example, by plating the cells in a culture plate that displays one or more substrate surfaces compatible with cell adhesion. When the one or more substrate surfaces contact the suspension of cells (e.g., suspension in a medium) introduced into the culture system, cell adhesion between the cells and the substrate surfaces may ensue. Accordingly, in certain embodiments cells are introduced into a culture system that features at least one substrate surface that is generally compatible with adherence of cells thereto, such that the plated cells can contact the said substrate surface, such embodiments encompass plating onto a substrate, which allows adherence of cells thereto.

Cells of the present disclosure may be identified and characterized by their expression of specific marker proteins, such as cell-surface markers. Detection and isolation of these cells can be achieved, for example, through flow cytometry, ELISA, and/or magnetic beads. Reverse-transcription polymerase chain reaction (RT-PCR) may be used to quantify cell-specific genes and/or to monitor changes in gene expression in response to differentiation. In certain embodiments, the marker proteins used to identify and characterize the cells are selected from the list consisting of c-Kit, Nanog, Sox2, Hey1, SMA, Vimentin, Cyclin D2, Snail, E-cadherin, Nkx2.5, GATA4, CD105, CD90, CD29, CD73, Wt1, CD34, CD45, and a combination thereof

### IX. Detecting a Genetic Signature

Particular embodiments concern the methods of detecting a genetic signature in an individual. In some embodiments, the method for detecting the genetic signature may include selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof, for example. The method for detecting the genetic signature may include fluorescent in situ hybridization, comparative genomic hybridization, arrays, polymerase chain reaction, sequencing, or a combination thereof, for example. The detection of the genetic signature may involve using a particular method to detect one feature of the genetic signature and additionally use the same method or a different method to detect a different feature of the genetic signature. Multiple different methods independently or in combination may be used to detect the same feature or a plurality of features.

### A. Single Nucleotide Polymorphism (SNP) Detection

Particular embodiments of the disclosure concern methods of detecting a SNP in an individual. One may employ any of the known general methods for detecting SNPs for detecting the particular SNP in this disclosure, for example. Such methods include, but are not limited to, selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, and a combination thereof.

In some embodiments of the disclosure, the method used to detect the SNP comprises sequencing nucleic acid material from the individual and/or using selective oligonucleotide probes. Sequencing the nucleic acid material from the individual may involve obtaining the nucleic acid material from the individual in the form of genomic DNA, complementary DNA that is reverse transcribed from RNA, or RNA, for example. Any standard sequencing technique may be employed, including Sanger sequencing, chain extension sequencing, Maxam-Gilbert sequencing, shotgun sequencing, bridge PCR sequencing, high-throughput methods for sequencing, next generation sequencing, RNA sequencing, or a combination thereof. After sequencing the nucleic acid from the individual, one may utilize any data processing software or technique to determine which particular nucleotide is present in the individual at the particular SNP.

In some embodiments, the nucleotide at the particular SNP is detected by selective oligonucleotide probes. The probes may be used on nucleic acid material from the individual, including genomic DNA, complementary DNA that is reverse transcribed from RNA, or RNA, for example. Selective oligonucleotide probes preferentially bind to a complementary strand based on the particular nucleotide present at the SNP. For example, one selective oligonucleotide probe binds to a complementary strand that has an A nucleotide at the SNP on the coding strand but not a G nucleotide at the SNP on the coding strand, while a different selective oligonucleotide probe binds to a complementary strand that has a G nucleotide at the SNP on the coding strand but not an A nucleotide at the SNP on the coding strand. Similar methods could be used to design a probe that selectively binds to the coding strand that has a C or a T nucleotide, but not both, at the SNP. Thus, any method to determine binding of one selective oligonucleotide probe over another selective oligonucleotide probe could be used to determine the nucleotide present at the SNP.

One method for detecting SNPs using oligonucleotide probes comprises the steps of analyzing the quality and measuring quantity of the nucleic acid material by a spectrophotometer and/or a gel electrophoresis assay; processing the nucleic acid material into a reaction mixture with at least one selective oligonucleotide probe, PCR primers, and a mixture with components needed to perform a quantitative PCR (qPCR), which could comprise a polymerase, deoxynucleotides, and a suitable buffer for the reaction; and cycling the processed reaction mixture while monitoring the reaction. In one embodiment of the method, the polymerase used for the qPCR will encounter the selective oligonucleotide probe binding to the strand being amplified and, using endonuclease activity, degrade the selective oligonucleotide probe. The detection of the degraded probe determines if the probe was binding to the amplified strand.

Another method for determining binding of the selective oligonucleotide probe to a particular nucleotide comprises using the selective oligonucleotide probe as a PCR primer, wherein the selective oligonucleotide probe binds preferentially to a particular nucleotide at the SNP position. In some embodiments, the probe is generally designed so the 3′ end of the probe pairs with the SNP. Thus, if the probe has the correct complementary base to pair with the particular nucleotide at the SNP, the probe will be extended during the amplification step of the PCR. For example, if there is a T nucleotide at the 3′ position of the probe and there is an A nucleotide at the SNP position, the probe will bind to the SNP and be extended during the amplification step of the PCR. However, if the same probe is used (with a T at the 3′ end) and there is a G nucleotide at the SNP position, the probe will not fully bind and will not be extended during the amplification step of the PCR.

In some embodiments, the SNP position is not at the terminal end of the PCR primer, but rather located within the PCR primer. The PCR primer should be of sufficient length and homology in that the PCR primer can selectively bind to one variant, for example the SNP having an A nucleotide, but not bind to another variant, for example the SNP having a G nucleotide. The PCR primer may also be designed to selectively bind particularly to the SNP having a G nucleotide but not bind to a variant with an A, C, or T nucleotide. Similarly, PCR primers could be designed to bind to the SNP having a C or a T nucleotide, but not both, which then does not bind to a variant with a G, A, or T nucleotide or G, A, or C nucleotide respectively. In particular embodiments, the PCR primer is at least or no more than 10, 11, 12, 13, 14, 15, 16, 17, 18, 19, 20, 21, 22, 23, 24, 25, 26, 27, 28, 29, 30, 31, 32, 33, 34,3 5, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or more nucleotides in length with 100% homology to the template sequence, with the potential exception of non-homology the SNP location. After several rounds of amplifications, if the PCR primers generate the expected band size, the SNP can be determined to have the A nucleotide and not the G nucleotide.

As described herein, a subject may be or have been genotyped as having one or more SNPs. For example, a subject may be or have been genotyped as having a SNP selected from rs1483185, rs1483186, and rs750170. SNPs disclosed herein may be described by one or more designations.

In some embodiments, a SNP is designated by a chromosomal location and one or more nucleotides. For example, a subject may be described as having the SNP chr8:127535470 T>A. In this example, such a description identifies the subject as having an A nucleotide (instead of the more common T nucleotide) at the chromosomal location chr8:127535470. In another example, a subject may be described as having the SNP chr13:49282062 C>A,T. In this example, such a description identifies the subject as having either an A nucleotide or a T nucleotide (instead of the more common C nucleotide) at the chromosomal location chr13:49282062.

In some embodiments, a SNP is designated by a Reference SNP (also “RefSNP” or “rs”) identifier. When a subject is described herein as having a particular SNP by use of an rs identifier, such a description is understood to encompass any nucleotide or sequence encompassed by the rs idenfieier. An rs identifier for a SNP may encompass one single nucleotide or may encompass two or more alternative nucleotides (i.e. two or more “alleles”). For example, a subject genotyped as having the SNP rs 111620024 describes a subject having a T allele at chromosomal position chr5:96662687, while a subject genotyped as having the SNP rs12653946 describes a subject having either an A or a T at chromosomal location chr5:1895715. Databases harboring information regarding SNPs (and other genomic variants) are known to the skilled artisan and include, for example, the Single Nucleotide Polymorphism Database (dbSNP), available on the World Wide Web at ncbi.nlm.nih.gov/snp, described in Smigielski E M, et al. dbSNP: a database of single nucleotide polymorphisms. Nucleic Acids Res. 2000 Jan. 1; 28(1):352-5, incorporated herein by reference in its entirety.

### B. Copy Number Variation Detection

Particular embodiments of the disclosure concern methods of detecting a copy number variation (CNV) of a particular allele. One can utilize any known method for detecting CNVs to detect the CNVs. Such methods include fluorescent in situ hybridization, comparative genomic hybridization, arrays, polymerase chain reaction, sequencing, or a combination thereof, for example. In some embodiments, the CNV is detected using an array. Array platforms such as those from Agilent, Illumina, or Affymetrix may be used, or custom arrays could be designed. One example of how an array may be used includes methods that comprise one or more of the steps of isolating nucleic acid material in a suitable manner from an individual suspected of having the CNV and, at least in some cases from an individual or reference genome that does not have the CNV; processing the nucleic acid material by fragmentation, labelling the nucleic acid with, for example, fluorescent labels, and purifying the fragmented and labeled nucleic acid material; hybridizing the nucleic acid material to the array for a sufficient time, such as for at least 24 hours; washing the array after hybridization; scanning the array using an array scanner; and analyzing the array using suitable software. The software may be used to compare the nucleic acid material from the individual suspected of having the CNV to the nucleic acid material of an individual who is known not to have the CNV or a reference genome.

In some embodiments, detection of a CNV is achieved by polymerase chain reaction (PCR). PCR primers can be employed to amplify nucleic acid at or near the CNV wherein an individual with a CNV will result in measurable higher levels of PCR product when compared to a PCR product from a reference genome. The detection of PCR product amounts could be measured by quantitative PCR (qPCR) or could be measured by gel electrophoresis, as examples. Quantification using gel electrophoresis comprises subjecting the resulting PCR product, along with nucleic acid standards of known size, to an electrical current on an agarose gel and measuring the size and intensity of the resulting band. The size of the resulting band can be compared to the known standards to determine the size of the resulting band. In some embodiments, the amplification of the CNV will result in a band that has a larger size than a band that is amplified, using the same primers as were used to detect the CNV, from a reference genome or an individual that does not have the CNV being detected. The resulting band from the CNV amplification may be nearly double, double, or more than double the resulting band from the reference genome or the resulting band from an individual that does not have the CNV being detected. In some embodiments, the CNV can be detected using nucleic acid sequencing. Sequencing techniques that could be used include, but are not limited to, whole genome sequencing, whole exome sequencing, and/or targeted sequencing.

### C. DNA Sequencing

In some embodiments, DNA may be analyzed by sequencing. The DNA may be prepared for sequencing by any method known in the art, such as library preparation, hybrid capture, sample quality control, product-utilized ligation-based library preparation, or a combination thereof. The DNA may be prepared for any sequencing technique. In some embodiments, a unique genetic readout for each sample may be generated by genotyping one or more highly polymorphic SNPs. In some embodiments, sequencing, such as 76 base pair, paired-end sequencing, may be performed to cover approximately 70%, 75%, 80%, 85%, 90%, 95%, 99%, or greater percentage of targets at more than 20×, 25×, 30×, 35×, 40×, 45×, 50×, or greater than 50× coverage. In certain embodiments, mutations, SNPS, INDELS, copy number alterations (somatic and/or germline), or other genetic differences may be identified from the sequencing using at least one bioinformatics tool, including VarScan2, any R package (including CopywriteR) and/or Annovar.

### D. RNA Sequencing

In some embodiments, RNA may be analyzed by sequencing. The RNA may be prepared for sequencing by any method known in the art, such as poly-A selection, cDNA synthesis, stranded or nonstranded library preparation, or a combination thereof. The RNA may be prepared for any type of RNA sequencing technique, including stranded specific RNA sequencing. In some embodiments, sequencing may be performed to generate approximately 10M, 15M, 20M, 25M, 30M, 35M, 40M or more reads, including paired reads. The sequencing may be performed at a read length of approximately 50 bp, 55 bp, 60 bp, 65 bp, 70 bp, 75 bp, 80 bp, 85 bp, 90 bp, 95 bp, 100 bp, 105 bp, 110 bp, or longer. In some embodiments, raw sequencing data may be converted to estimated read counts (RSEM), fragments per kilobase of transcript per million mapped reads (FPKM), and/or reads per kilobase of transcript per million mapped reads (RPKM). In some embodiments, one or more bioinformatics tools may be used to infer stroma content, immune infiltration, and/or tumor immune cell profiles, such as by using upper quartile normalized RSEM data.

### E. Proteomics

In some embodiments, protein may be analyzed by mass spectrometry. The protein may be prepared for mass spectrometry using any method known in the art. Protein, including any isolated protein encompassed herein, may be treated with DTT followed by iodoacetamide. The protein may be incubated with at least one peptidase, including an endopeptidase, proteinase, protease, or any enzyme that cleaves proteins. In some embodiments, protein is incubated with the endopeptidase, LysC and/or trypsin. The protein may be incubated with one or more protein cleaving enzymes at any ratio, including a ratio of pg of enzyme to pg protein at approximately 1:1000, 1:100, 1:90, 1:80, 1:70, 1:60, 1:50, 1:40, 1:30, 1:20, 1:10, 1:1, or any range between. In some embodiments, the cleaved proteins may be purified, such as by column purification. In certain embodiments, purified peptides may be snap-frozen and/or dried, such as dried under vacuum. In some embodiments, the purified peptides may be fractionated, such as by reverse phase chromatography or basic reverse phase chromatography. Fractions may be combined for practice of the methods of the disclosure. In some embodiments, one or more fractions, including the combined fractions, are subject to phosphopeptide enrichment, including phospho-enrichment by affinity chromatography and/or binding, ion exchange chromatography, chemical derivatization, immunoprecipitation, co-precipitation, or a combination thereof. The entirety or a portion of one or more fractions, including the combined fractions and/or phospho-enriched fractions, may be subject to mass spectrometry. In some embodiments, the raw mass spectrometry data may be processed and normalized using at least one relevant bioinformatics tool.

### F. Detection Kits and Systems

One can recognize that based on the methods described herein, detection reagents, kits, and/or systems can be utilized to detect a SNP and/or CNV related to the genetic signature for diagnosing or prognosing an individual (the detection either individually or in combination). The reagents can be combined into at least one of the established formats for kits and/or systems as known in the art. As used herein, the terms “kits” and “systems” refer to embodiments such as combinations of at least one SNP detection reagent, for example at least one selective oligonucleotide probe, and at least one CNV detection reagent, for example at least one PCR primer. The kits could also contain other reagents, chemicals, buffers, enzymes, packages, containers, electronic hardware components, etc. The kits/systems could also contain packaged sets of PCR primers, oligonucleotides, arrays, beads, or other detection reagents. Any number of probes could be implemented for a detection array. In some embodiments, the detection reagents and/or the kits/systems are paired with chemiluminescent or fluorescent detection reagents. Particular embodiments of kits/systems include the use of electronic hardware components, such as DNA chips or arrays, or microfluidic systems, for example. In specific embodiments, the kit also comprises one or more therapeutic or prophylactic interventions in the event the individual is determined to be in need of.

### Examples

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—an Unbiased Genome Wide Association Study Correlates Melanoma Patient Germline SNPs to an Inflammatory Gene Signature

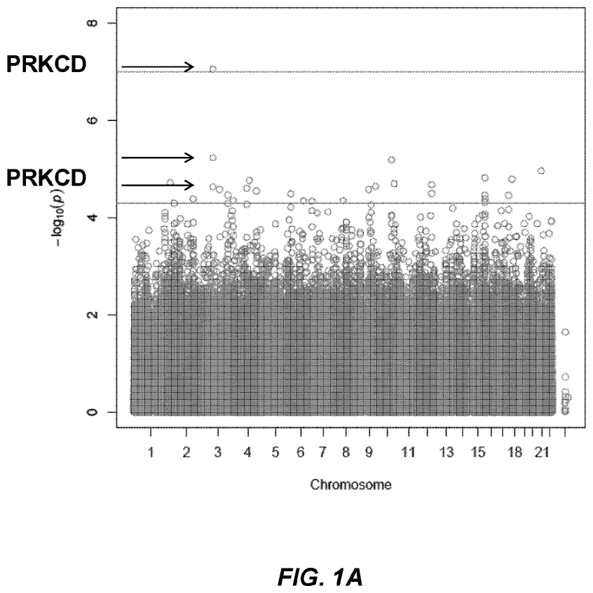

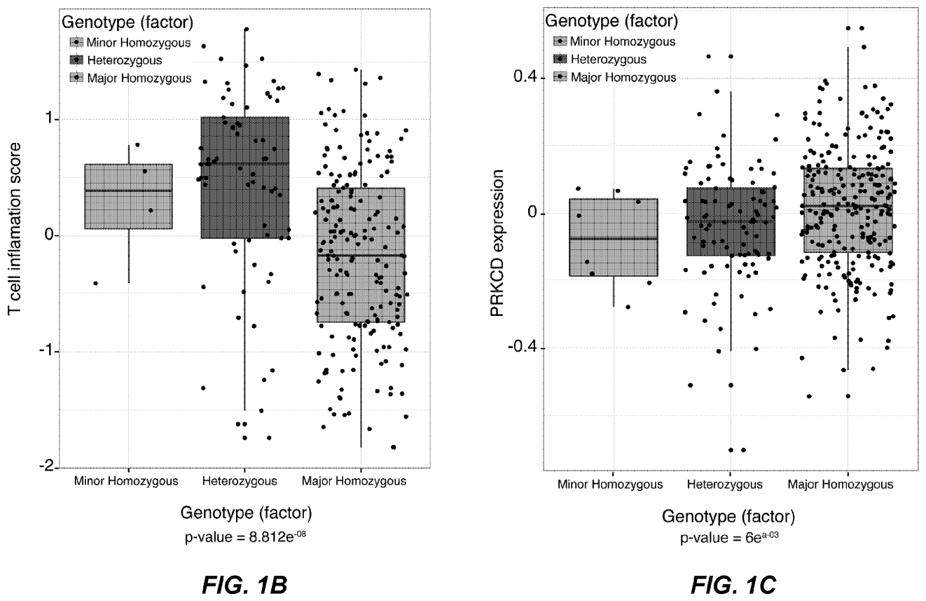

The degree of endogenous immune cell infiltration in solid tumors can be predictive of response to PD-1/PD-Li blockade yet is dramatically variable between individual patients. The underlying mechanisms determining the degree of a T cell-inflamed versus non-T cell-inflamed tumor microenvironment are not fully understood 5. To identify potential germline genetic variants that might influence immune infiltration in metastatic melanoma patients, the inventors integrated tumor immune gene signature RNAseq data with germline single nucleotide polymorphism (SNP) data from 258 patients in the TCGA melanoma dataset (FIG. 5A). A genome wide association study (GWAS) was performed in search of individual germline SNPs associated with the magnitude of the immune gene signature score. This approach identified a trio of neighbouring SNPs located within the same gene on chromosome 3 (FIG. 1A). The minor allele of the top GWAS hit (SNP rs1483185, located within the PKCd gene) was positively associated with a higher magnitude T cell gene signature score (p=8.812e-08, Bonferroni corrected <0.05) (FIG. 1B). Utilizing a lymphoblastoid cell line (LCL) eQTL dataset 6, the inventors interrogated SNP rs1483185 and determined that the minor allele was associated with decreased expression of PRKCD itself (Beta=0.0551, p=0.006) (FIG. 1C and Table 1).

TABLE 1

| SNP | Gene | Beta | p-value |
|---|---|---|---|
| rs1483185 | ENSG00000163932 | 0.05509 | 0.00646 |
| rs1483186 | ENSG00000163932 | 0.05054 | 0.01803 |
| rs750170 | ENSG00000163932 | 0.5471 | 0.00605 |

To extend these results, the inventors also examined the relationship of the top SNP with a more expansive T cell gene signature consisting of 160 genes 7, which similarly showed that the minor allele positively associated with an increase in the immune gene signature score (FIG. 5B).

To go beyond melanoma, the lung squamous TCGA dataset was examined and the minor allele of SNP rs1483185 was again correlated with an increase in the 160 gene signature score set (FIG. 5C). Interestingly, the two other SNPs (rs1483186, rs750170) located within the PKCd gene, also had minor alleles that associated with an increase in immune gene signature scores (FIGS. 5D-5E) and correlated with lower PRKCD expression in the LCL eQTL dataset (FIG. 5B). Collectively, these data indicate that reduced host PKCd gene expression is associated with increased T cell-based inflammation in the tumor microenvironment.

### Example 2—Characterization of the Effect of Host PKCδ Loss on B16 Tumor Growth and Tumor Immunity in a Knock Out Mouse Model

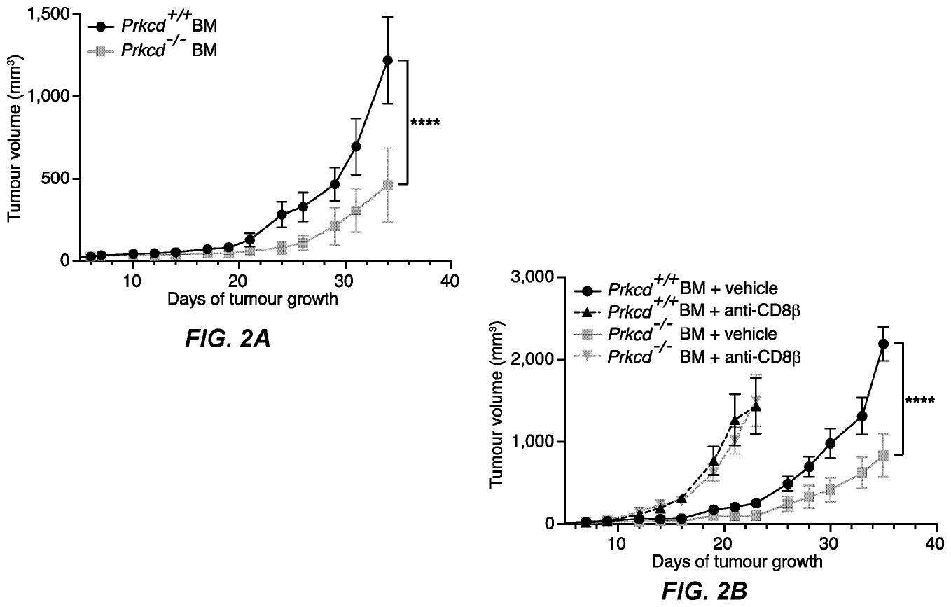

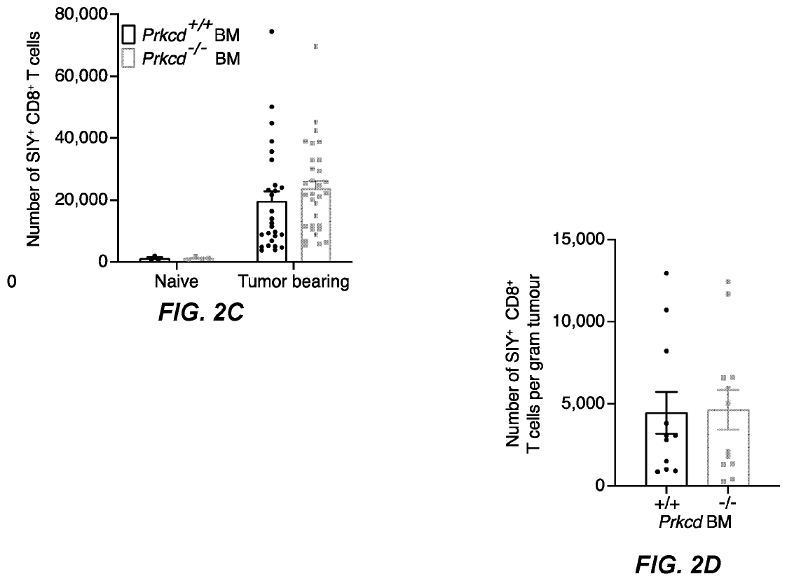

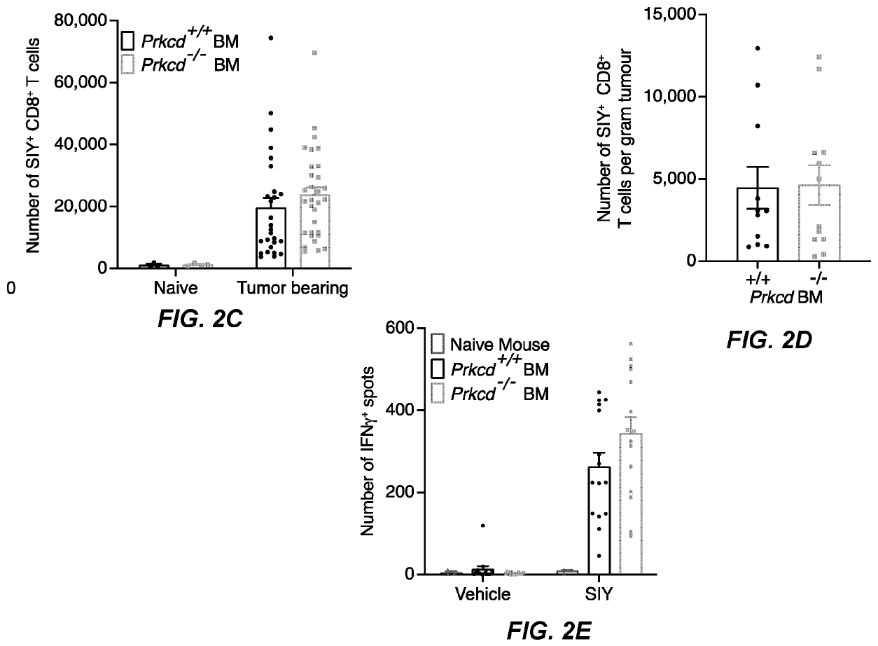

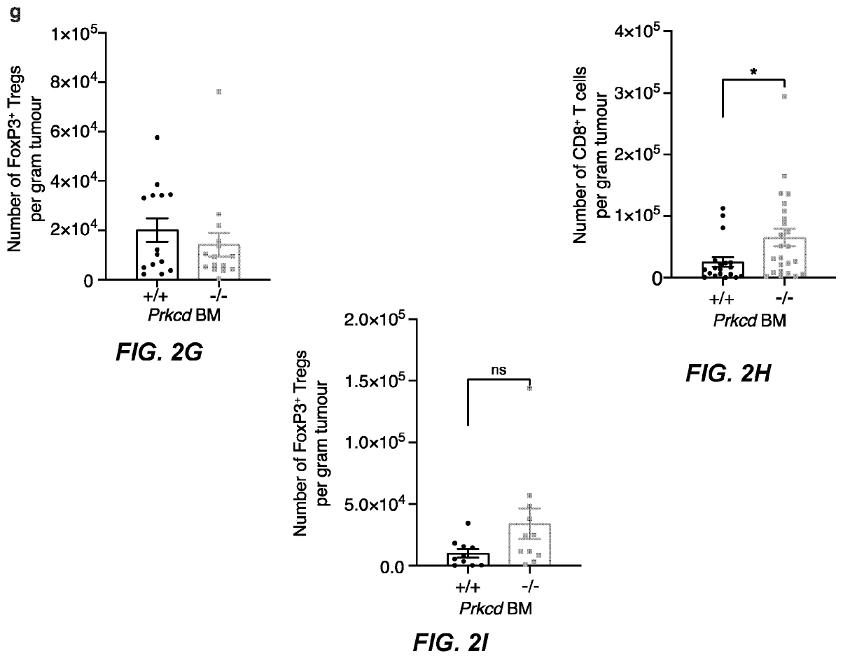

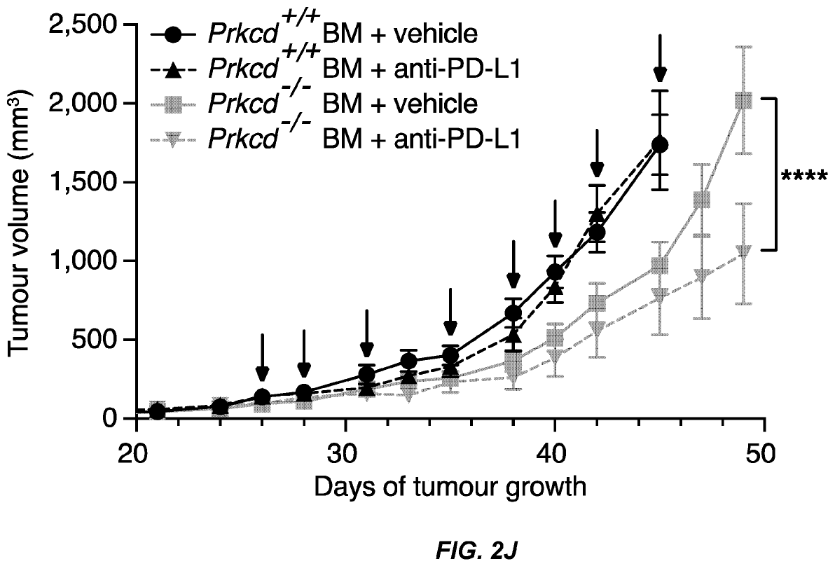

Previous studies have linked genetic loss of PKCδ in humans with autoimmunity, including familial lupus^{8,9}, and an autoimmune phenotype has been reported in PKCδ gene-targeted mice¹⁰. The inventors therefore pursued the hypothesis that deletion of the PKCδ gene in host immune cells might augment anti-tumor immunity. To test this notion, the inventors generated a bone marrow (BM) chimera utilizing Prkcd^{-/-} mice backcrossed to the C57BL/B6 background¹¹. This allowed us to isolate PKCδ loss to the hematopoietic compartment while overcoming previously observed breeding difficulties with these animals¹². Mice were implanted with B16-SIY melanoma tumor cells, which allowed measuring of CD8⁺ T cell responses against a model antigen. The inventors observed that tumors grew significantly more slowly in mice engrafted with Prkcd^{-/-} BM compared to animals engrafted with Prkcd^{+/+} BM (FIG. 2A). To assess whether these growth differences were due to altered host T cell immunity, the inventors treated mice with a CD8β depleting antibody and tumor growth differences were eliminated, arguing that the delay in the absence of host PKCδ was due to improved T cell-mediated tumor control (FIG. 2B).

The potential mechanism by which host anti-tumor immunity was augmented with the loss of hematopoietic cell PKCδ was pursued. Bone marrow engrafted mice showed no differences in CD8⁺ T cell populations at baseline, although there was an increase in the total number of FoxP3⁺ T regulatory (Treg) cells in the PKCδ KO setting (FIGS. 6A-6B), consistent with counter-regulation of heightened endogenous immunity. The magnitude of priming of tumor antigen-specific CD8⁺ T cells was assessed using SIY-K^{b} pentamer staining and IFN-γ ELISPOT assays. At day 10 following tumor implantation, the numbers of SIY-specific CD8⁺ T cells was comparable in the spleens of Prkcd^{+/+} and Prkcd^{-/-} BM-engrafted mice, and similar numbers were also observed within the tumor microenvironment (FIG. 2C-2D, FIG. 6C). Comparable functionality of those cells was also inferred by similar IFN-γ ELISPOT results in WT and KO engrafted hosts (FIG. 2E). Moreover, bulk T cell populations in the spleen and tumor of day 10 tumor-bearing mice revealed no differences in total number of splenic or tumor resident CD3⁺, CD8⁺, conventional CD4⁺ T cells, or tumor resident FoxP3⁺ T reg cells (FIGS. 2F-2G, FIG. 6D-6E), although an increased number and percentage of FoxP3⁺ Treg cells was observed in the spleen, as had been seen in the absence of tumor implantation. Together, these data indicate that improved immune-mediated tumor control in the absence of host PKCδ is not due to increased priming of tumor antigen-specific CD8⁺ T cells or through decreased numbers of FoxP3⁺ Treg cells.

To evaluate whether improved anti-tumor immunity might be reflected over time at the level of the tumor microenvironment, the inventors characterized the immune infiltrate at later time points as the tumors grew, focusing on day 35. In fact, tumors from Prkcd^{-/-} BM-engrafted animals analyzed at day 35 showed more CD3⁺ and CD8⁺ T cells per gram of tumor (FIG. 2B, FIGS. 6F-6H). This phenomenon was specific to the CD8⁺ T cell compartment, as PKCδ KO samples did not have increased numbers per gram of tumor of FoxP3⁺ T reg cells, conventional CD4⁺ T cells, and PD-1⁺ T cells (FIG. 21, FIG. 6G). Splenic CD8⁺ and CD3⁺ T cell populations remained unchanged at this time point indicating the increase in CD8⁺ T cells was localized to the tumor microenvironment (FIG. 6I-6J). Thus, like patients possessing a hypomorphic allele of the PKCδ gene, tumors in PKCδ-deficient mice demonstrated greater T cell infiltration in the tumor microenvironment over time.

With improved spontaneous immune-mediated tumor control and an increased accumulation of tumor-infiltrating CD8⁺ T cells, the inventors hypothesized PKCδ KO hosts may show improved efficacy of PD-i/PD-L1 blockade. To this end, Prkcd^{-/-} versus Prkcd^{+/+} BM-engrafted mice were implanted with B16-SIY cells and treated with anti-PD-L1 antibody 3× per week beginning on day 26. Indeed, Prkcd^{-/-} BM-engrafted mice showed improved anti-PD-L1 efficacy compared to Prkcd^{+/+} BM-engrafted animals (FIG. 2J), which was accompanied by significantly improved overall survival (FIG. 6K). Thus, loss of PKCδ in the hematopoietic compartment caused a CD8⁺ T cell-dependent delay in tumor outgrowth, which could be further augmented with the addition of anti-PD-L1 antibody treatment

### Example 3—scRNAseq Reveals Macrophage Phenotypic Changes Including Hematopoietic Loss of PKCδ Alters M1/M2 Ratio in the Macrophage Compartment in the Tumor Microenvironment

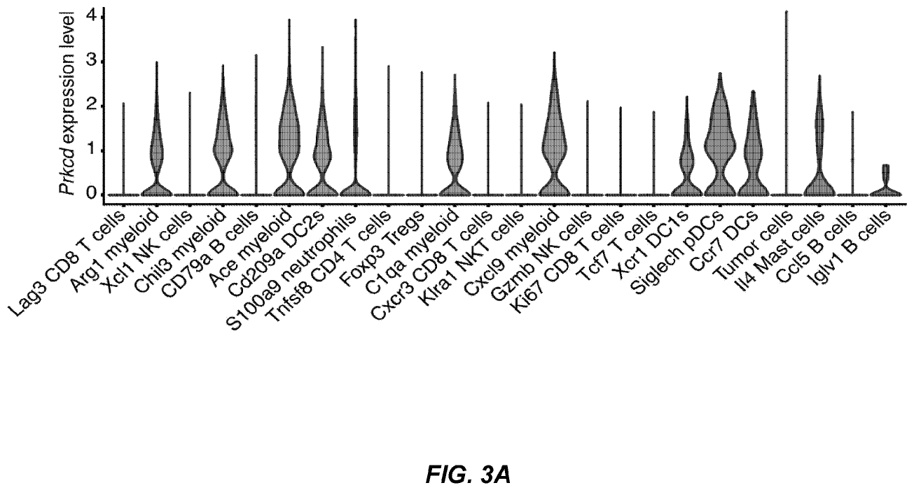

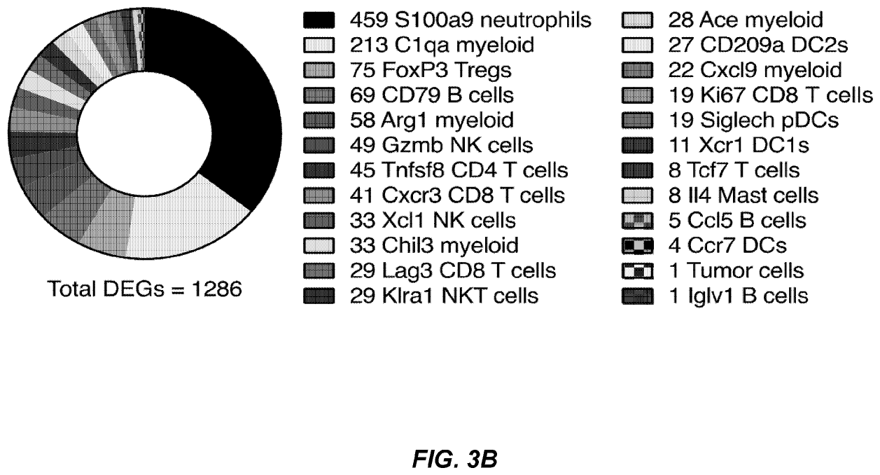

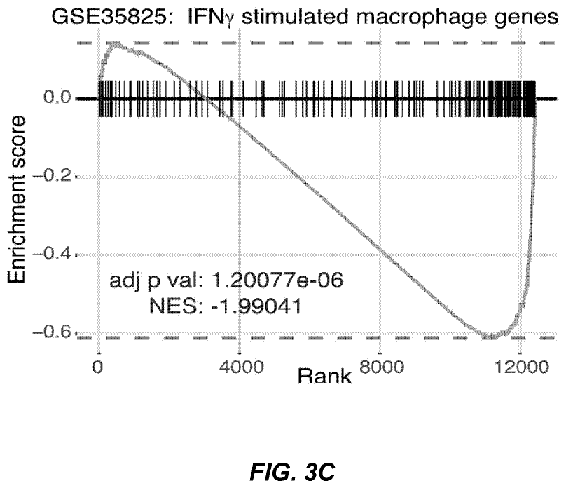

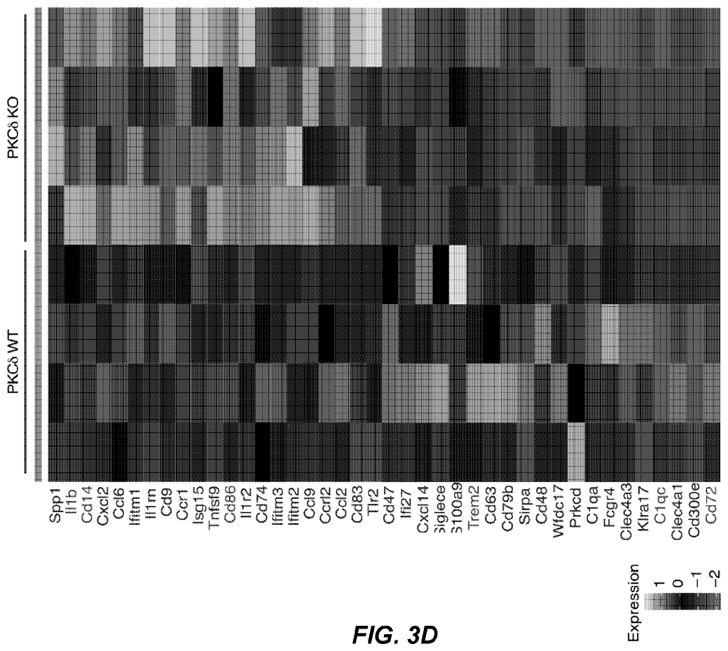

To investigate the mechanism underlying the improvement in anti-tumor immunity at the level of the tumor microenvironment, the inventors isolated live tumor-infiltrating dsRed⁻ CD45⁺cells for single cell RNA sequencing (scRNAseq). Cells were isolated by flow cytometry after 26 days of tumor growth, which is the time frame in which the Prkcd^{+/+} and Prkcd^{-/-} BM-engrafted mouse tumor growth curves just began to separate. Utilizing the Seurat package, sequencing data was analyzed and a UMAP projection was created revealing a total of 24 uniquely identifiable clusters (FIG. 7). After clusters were classified by cell type, each cluster was labeled by a cluster-defining gene which is uniquely expressed for that cluster. To narrow down which cells may be directly affected by PKCδ deficiency, Prkcd expression itself was probed in the WT samples. High Prkcd expression was mainly identified in the various myeloid cell subsets (FIG. 3A), in agreement with previously published datasets in the Immunological Genome Project database¹³. Differentially expressed genes (DEGs) between WT and PKCδ KO samples with a false discovery rate (fdr)≤0.05 and a log fold-change magnitude ≥0.25 were then identified. Clusters identified by macrophage and neutrophil lineage markers comprised over 50% of all DEGs detected (FIG. 3B). Because Prkcd expression was expressed higher in macrophages at baseline and given their defined role in anti-tumor immunity, the macrophage subsets were focused upon for further analysis.

Tumor-associated macrophages (TAMs) can generally be classified as either having a pro-inflammatory “M1” phenotype, or the immunosuppressive “M2” phenotype and the status of these macrophages has been shown to directly influence the immunological microenvironment including influencing the numbers and status of T cells^{14,15}. Gene set enrichment analysis (GSEA) of the C1qa⁺ macrophage cluster revealed an enrichment of M1-type genes, including multiple transcripts related to IFN-γ signaling¹⁶, in the PKCδ KO context (FIG. 3C, Table 2).

TABLE 2
GSEA results showing the top 10 macrophage related pathways along with the Benjamini-Hochberg adjusted p-value
(padj), enrichment score (ES), and normalized enrichment score (NES). Red text highlights pathway of interest.

| Pathway Name | pAdj | ES | NES |
|---|---|---|---|
| GSE14769_UNSTIM_VS_60 MIN_LPS_BMDM_DN | 1.57E−08 | −0.73766 | −2.40338 |
| GSE14769_UNSTIM_VS_80 MIN_LPS_BMDM_DN | 1.57E−08 | −0.72894 | −2.37456 |
| GSE14769_UNSTIM_VS_40 MIN_LPS_BMDM_DN | 1.57E−08 | −0.69646 | −2.27368 |
| GSE14769_UNSTIM_VS_120 MIN_LPS_BMDM_DN | 1.57E−08 | −0.65705 | −2.14505 |
| GSE36891_UNSTIM_VS_POLYIC_TLR3_STIM_PERITONEAL_MACROPHAGE_UP | 1.57E−08 | −0.7542 | −2.24173 |
| GSE26343_UNSTIM_VS_LPS_STIM_NFAT5_KO_MACROPHAGE_DN | 1.57E−08 | −0.68662 | −2.23933 |
| GSE26343_UNSTIM_VS_LPS_STIM_MACROPHAGE_UP | 4.71E−08 | −0.64229 | −2.09841 |
| GSE35825_IFNA_VS_IFNG_STIM_MACROPHAGE_UP | 1.55E−07 | −0.67098 | −2.09819 |
| GSE35825_UNTREATED_VS_IFNG_STIM_MACROPHAGE_UP | 1.20E−06 | −0.61102 | −1.99041 |

Further examination of the top immune-related differentially expressed genes revealed upregulation of CD14 (which is a coreceptor for multiple TLRs), and IL-1β (FIG. 3D)¹⁷. In contrast, multiple M2-related genes were expressed at higher levels in the WT context, including Trem2, C1qc, and CD72¹⁸⁻²⁰. (FIG. 3D). Taken together, these results suggest that genetic loss of PKCδ leads to a shift in the TAM expression profile from an immunosuppressive M2 phenotype to a pro-inflammatory Ml phenotype

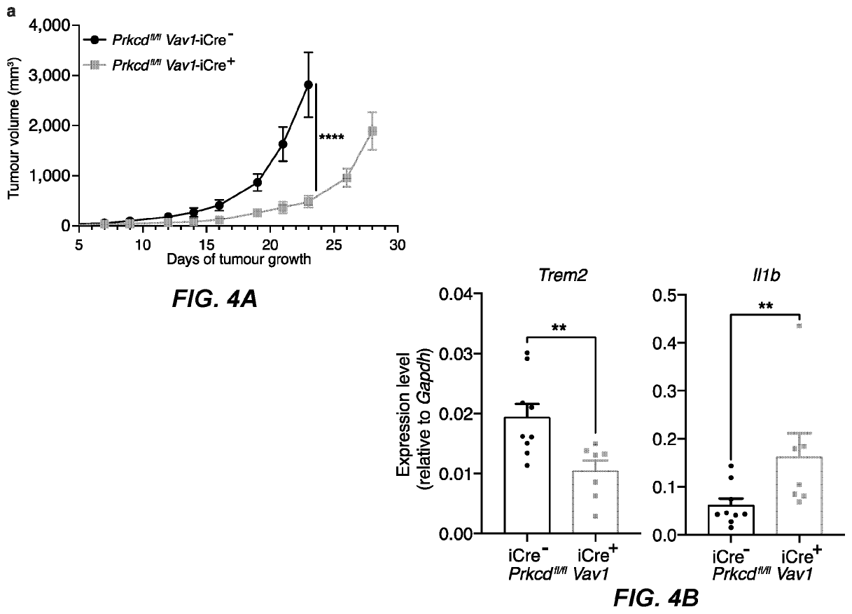

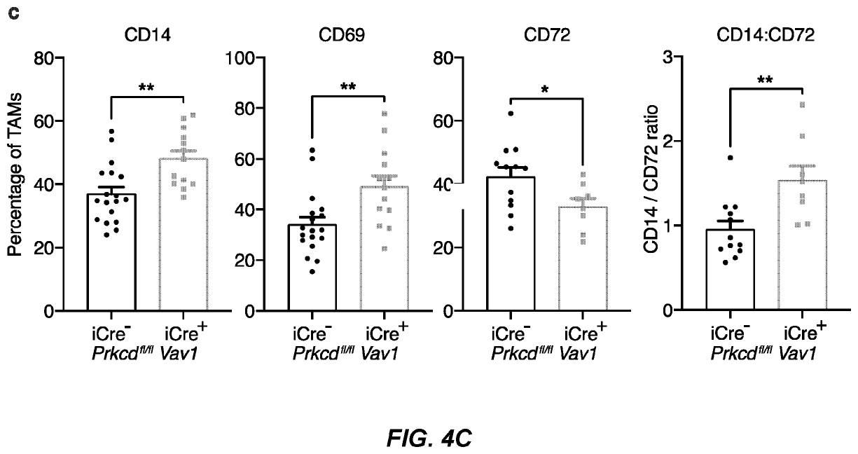

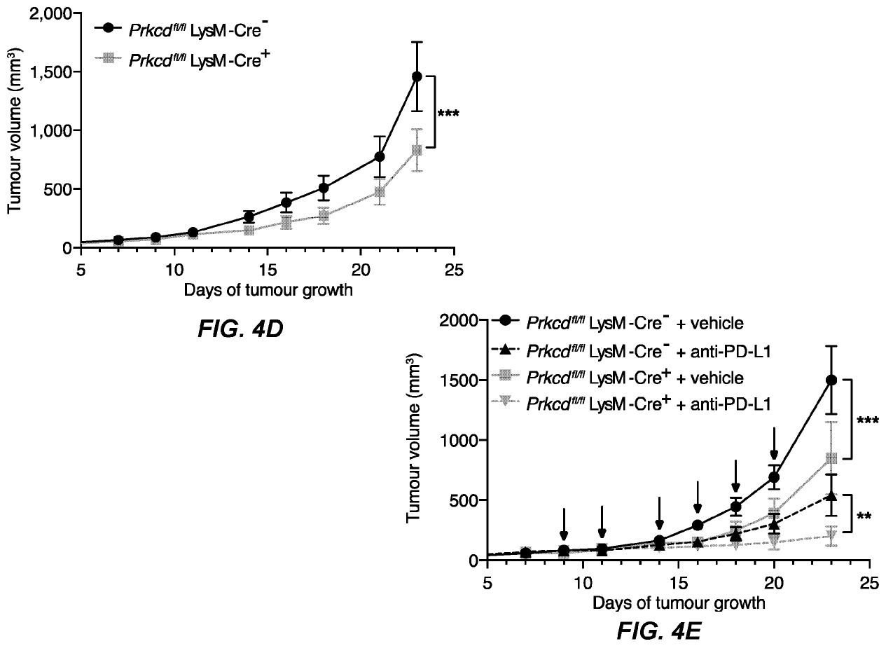

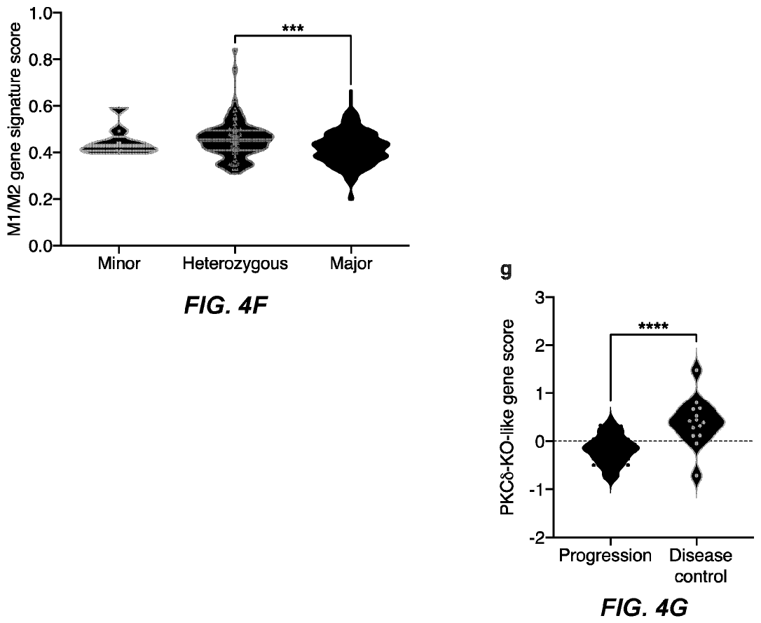

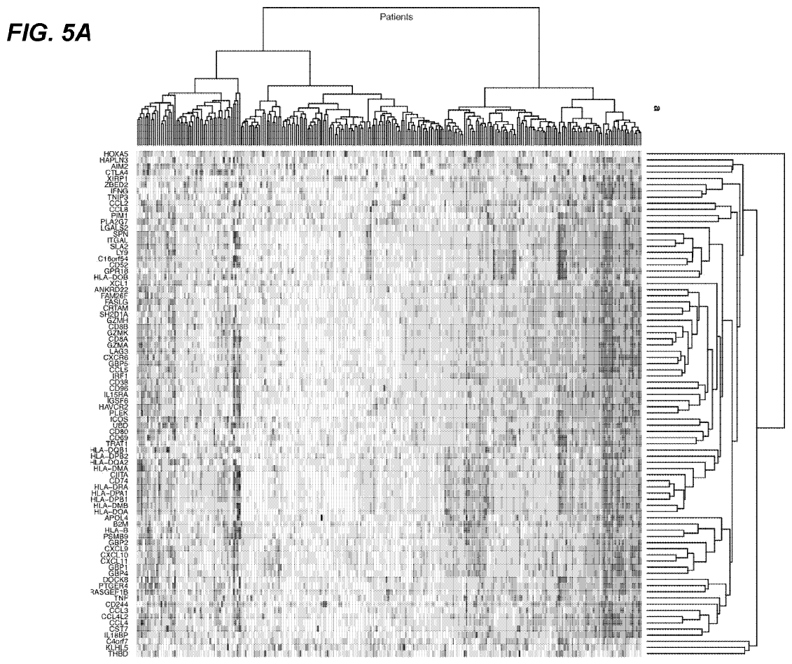

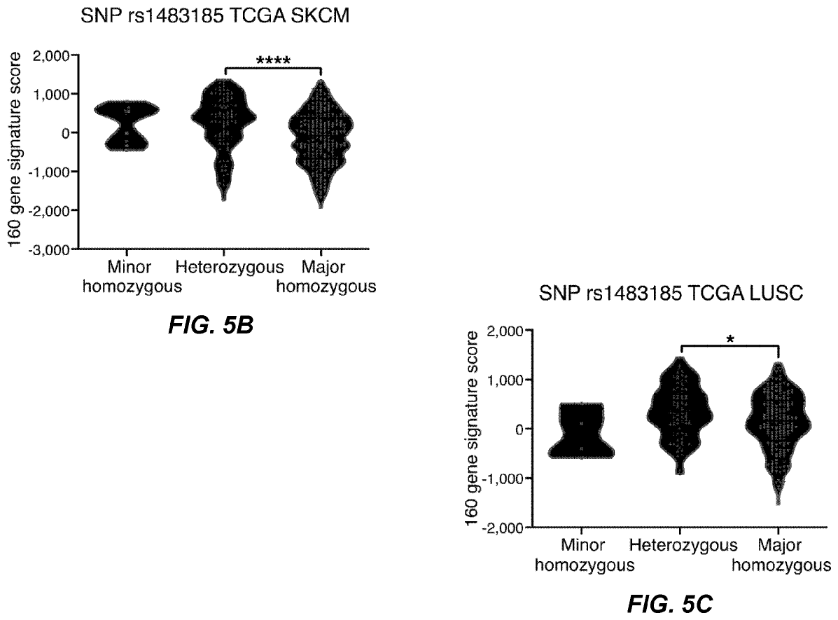

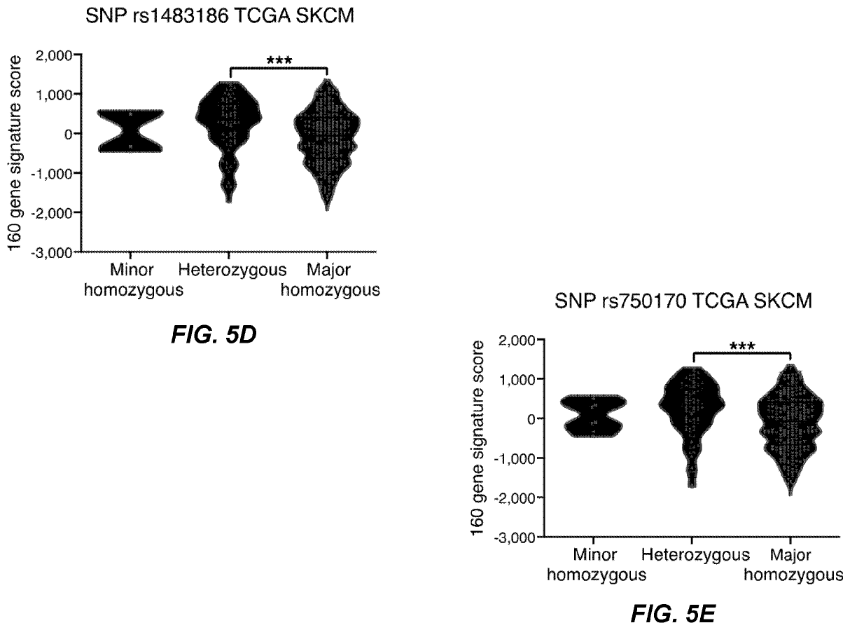

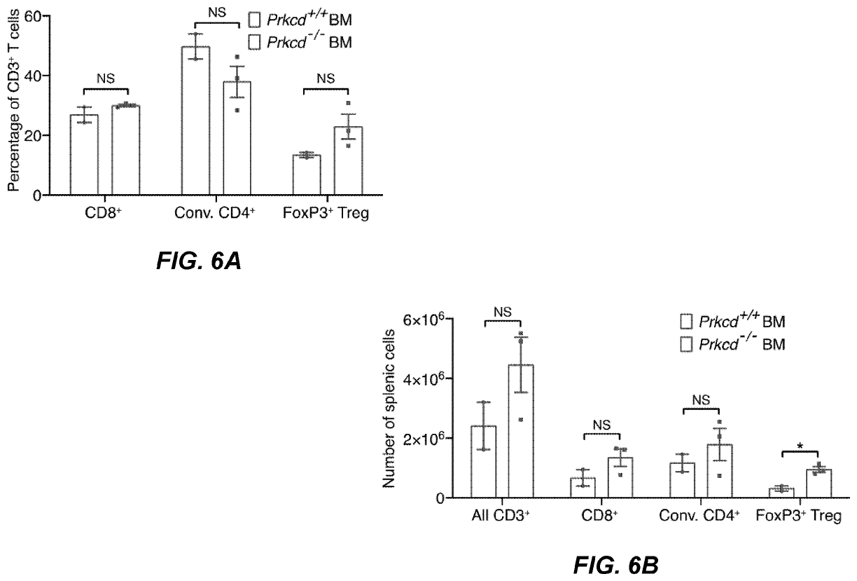

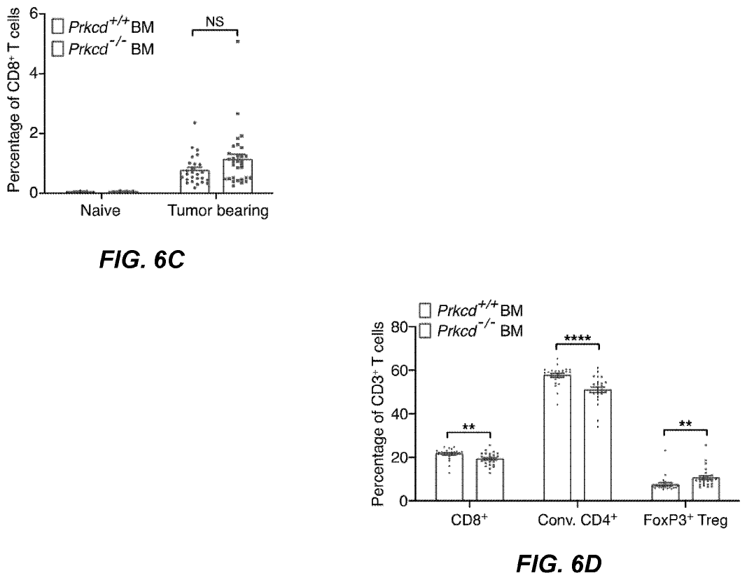

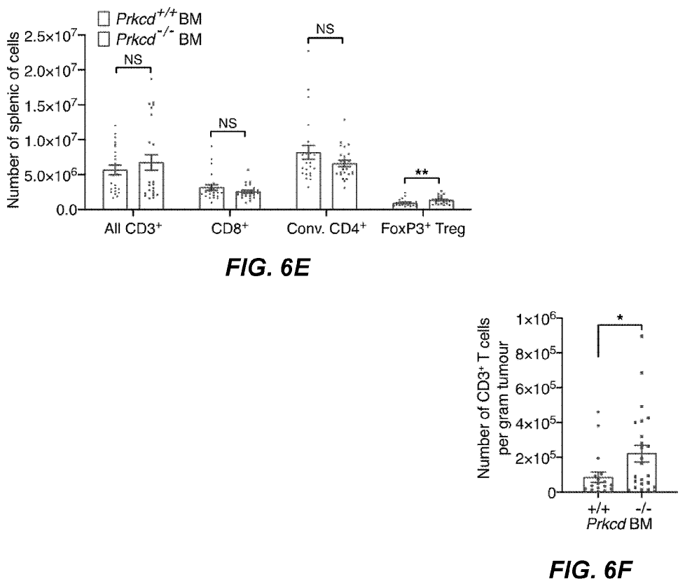

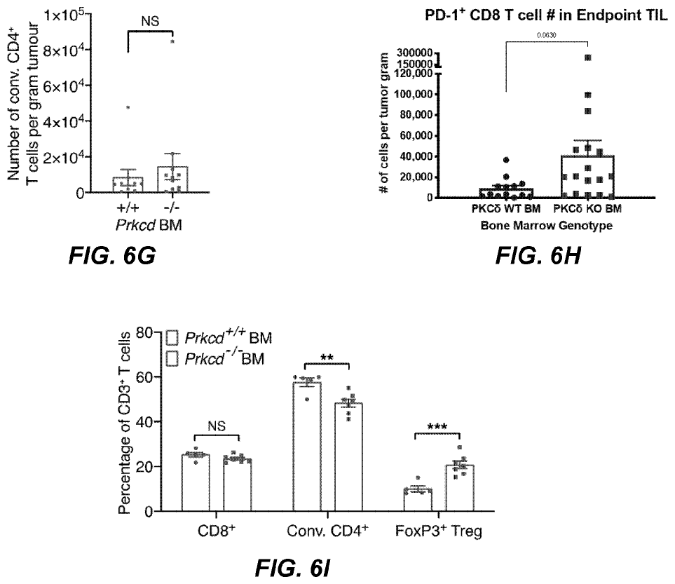

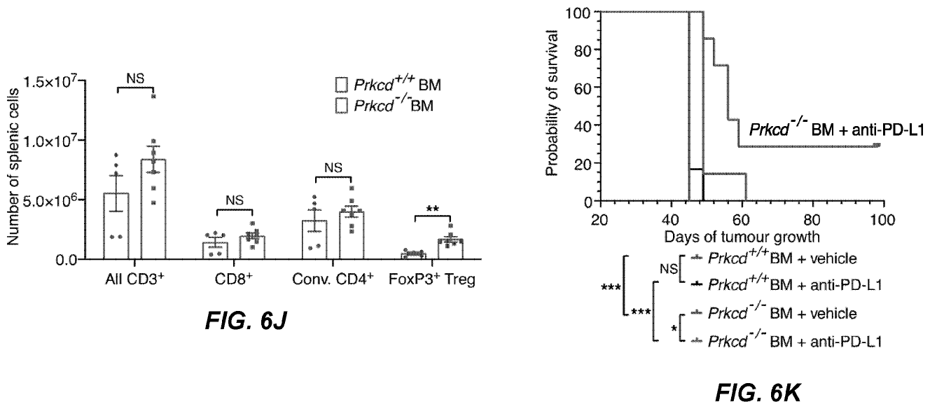

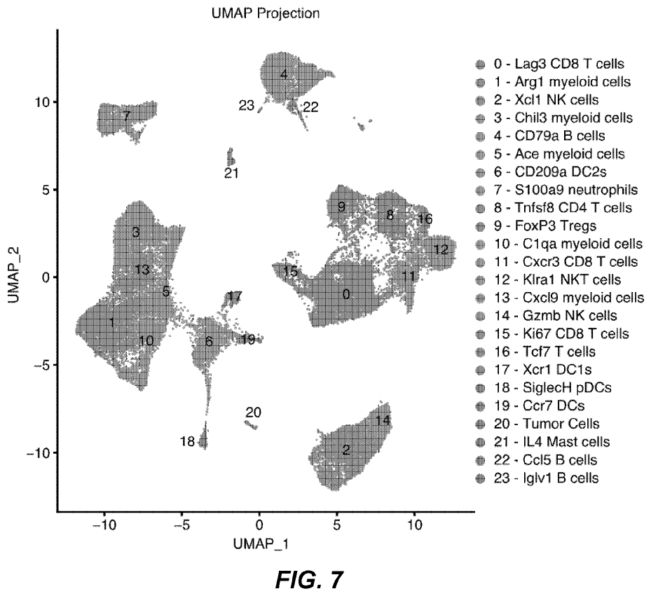

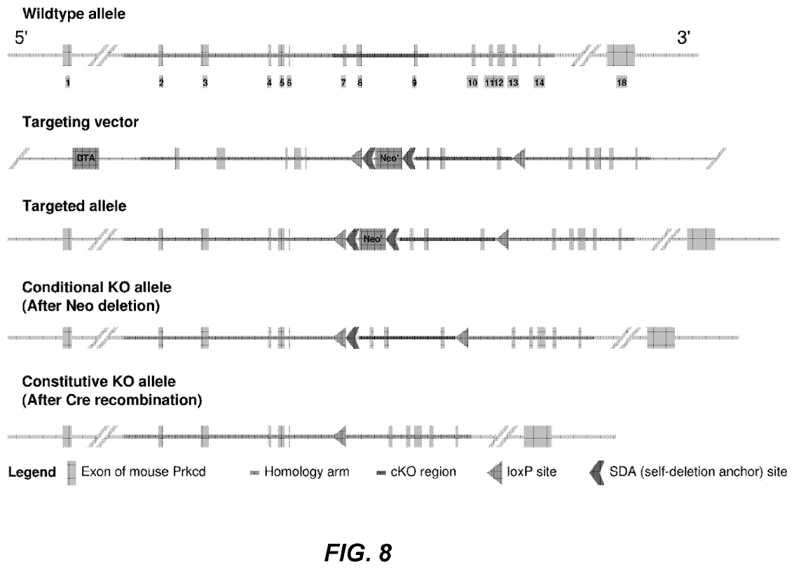

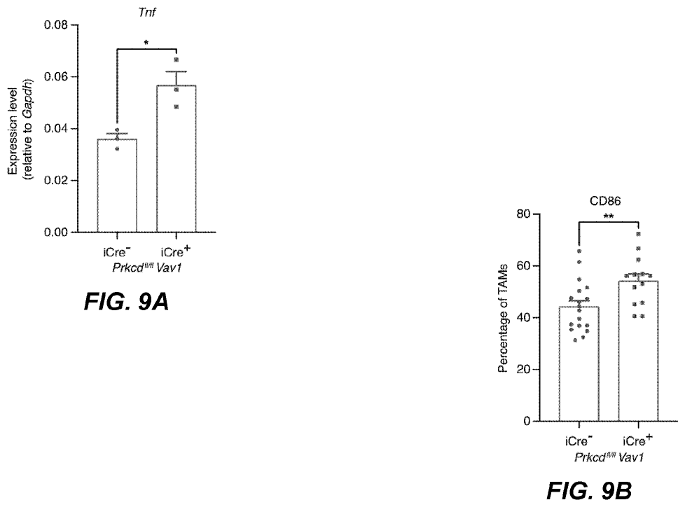

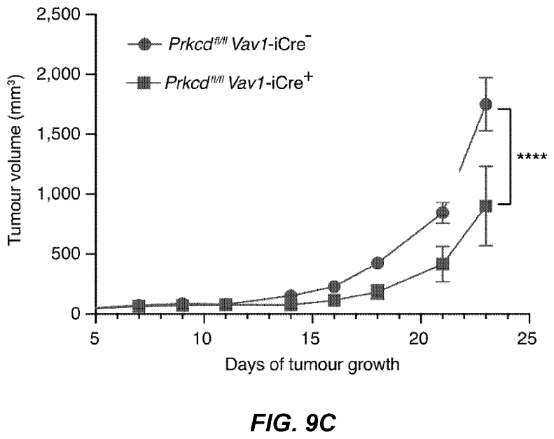

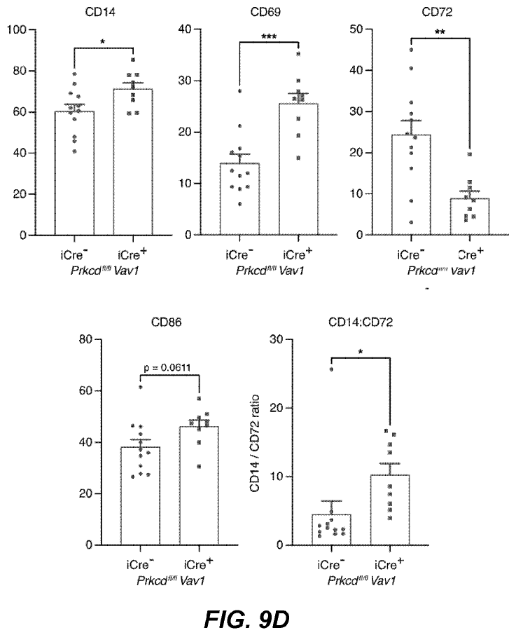

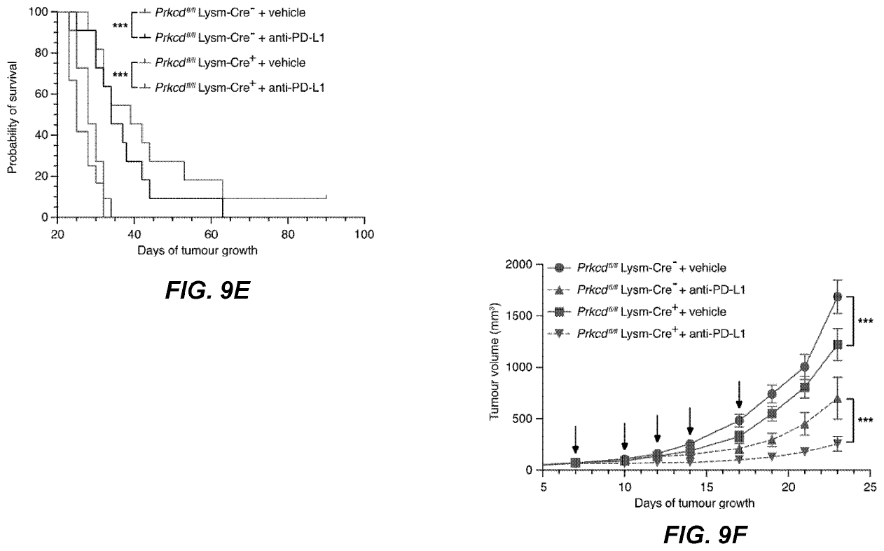

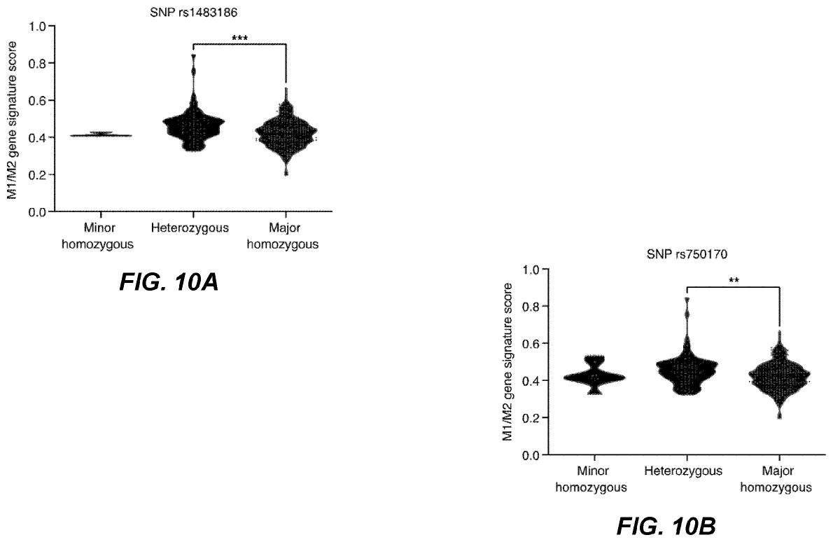

To enable targeted deletion of the PKCδ gene within the macrophage lineage, the inventors generated a conditional KO mouse model. Exons 7-9 were flanked by LoxP sites in C57BL/6 ES cells, which were used to generate genetically engineered mice (FIG. 8). Prkcd^{fl/fl} mice were first intercrossed with Vavl-iCre transgenic (tg) mice, to generate mice lacking PKCS in all hematopoietic cells. When B16-SIY tumors were implanted in vivo, tumor growth was slower in Prkcd^{fl/fl}/Vavl-iCre⁺ mice compared to Cre⁻ controls (FIG. 4A), confirming the previous results using the bone marrow chimera model. The inventors then examined the inflammatory profile of tumor-infiltrating macrophages by probing for representative M1 and M2 genes in TAMs isolated from Prkcd^{fl/fl}/Vavl-iCre⁺ and Prkcd^{fl/fl}/Vavl-iCre⁻ endpoint tumors. The inventors found that the characteristic M2 gene Trem2 was more highly expressed in macrophages isolated from tumors in Cre⁻ mice, and that the characteristic M1 genes Illb and Tnf were more highly expressed in macrophages from tumors in Cre⁺ mice (FIG. 4C, FIG. 9A)¹⁶. As a parallel approach, the inventors evaluated surface expression of CD14 and CD72, as these markers were top hits and have been used to characterize M1 versus M2 differentiation states, respectively 18,21. Indeed, the inventors found that Cre⁺ animals had a higher percentage of TAMs expressing CD14 as well as the known activation markers CD69 and CD86, while Cre⁻ macrophages had a higher percentage of CD72-expressing TAMs (FIG. 4C, FIG. 9B)^{15,22}. The inventors confirmed these data by crossing Vavl-iCre mice to a second, independently derived PKCδ conditional KO mouse line and saw similar results, both in terms of the tumor growth phenotype and TAM marker expression (FIGS. 9C-9D). Together, these data confirm that the genetic loss of PKCδ in hematopoietic cells leads to improved immune-mediated tumor growth control, which is associated with a shift in macrophages from an M2-like towards an MI-like phenotype within the tumor microenvironment.

To evaluate loss of PKCδ exclusively in the macrophage lineage, the inventors crossed Prkcd^{fl/fl} mice to LysM-Cre tg mice. The inventors found that growth of B16-SIY tumors was significantly delayed in Prkcd^{fl/fl}/LysM-Cre⁺ mice when compared to Cre⁻ littermate controls, phenocopying the results in the Vavl-Cre tg system (FIG. 4D). The inventors then investigated whether PKCδ loss selectively in myeloid cells could synergize with PD-1/PD-L1 blockade. Indeed, the inventors found a significant additional therapeutic benefit of anti-PD-L1 therapy when administered to Prkcd^{fl/fl}/LysM-Cre⁺ mice compared to control mice, including a complete response (FIG. 4E, FIG. 9E). When LysM-Cre was crossed to a second conditional KO founder line, similar synergistic results were observed (FIG. 9F). The inventors conclude that loss of PKC6 exclusively in the myeloid compartment is sufficient to delay tumor growth and synergize with anti-PD-L 1 therapy.

### Example 4—PKCδ Loss in the LysM Compartment is Sufficient for Tumor Growth Delay and Improved Response to Checkpoint Inhibition Showing Clinical Relevance

Inasmuch as the mouse model data indicated that PKCδ loss predominantly altered the ratio of M1/M2 phenotype within TAMs, the inventors examined the human melanoma TCGA dataset in pursuit of a similar relationship. To this end, the inventors utilized a previously validated list of M1 and M2 genes to create an M1 and an M2 signature score for each patient sample²³, then converted this into an M1/M2 ratio score. The M1 and M2 genes include: ACHE, ADAMDEC1, APOBEC3A, APOL3, APOL6, AQP9, ARRB1, CCL19, CCL5, CCL8, CCR7, CD38, CD40, CHI3L1, CLIC2, CXCL10, CXCL11, CXCL13, CXCL9, CYP27B1, DHX58, EBI3, GGT5, HESX1, IDO1, IF1I44L, IL2RA, KIAA0754, KYNU, LAG3, LAMP3, LILRA3, LILRB2, NOD2, PLA1A, PTGIR, RASSF4, RSAD2, SIGLECI, SLAMFI, SLC15A3, SLC2A6, SOCS1, TLR7, TLR8, TNFAIP6, TNIP3, and TRPM4. Interestingly, the inventors found that all 3 germline SNPs identified in the PKCδ gene with minor alleles that correlated with decreased PRKCD expression, also correlated with a higher M1/M2 gene expression ratio score (FIG. 4F, FIGS. 10A-10B).

The inventors then sought to determine whether a baseline PKCδ KO-related gene signature might predict clinical outcome to checkpoint blockade immunotherapy. To do this the inventors created a gene signature using the C1qa⁺ cluster DEGs that were increased in PKCδ KO mouse samples, and identified a human ortholog gene score. Such genes include NUFIPI, FI0, PIM3, PEDS1, CEBPB, CREM, CSF2RB, OSM, SMOX, LGALS1, CSRNP1, AHNAK, CLEC4E, RAB20, MALT1, NINJI, THBS1, PTAFR, AHR, CD83, PLBD1, CARD19, JDP2, ACOD1, PIM1, RGCC, CCL23, CCL15, CCL15-CCL14, CCL2, CLEC6A, HLA-DQB1, GOT1, RAB1lFIPI, CALMI, MAP4K4, FEMIC, F13A1, CDKN1A, SLC7A11, C5AR1, GNA13, SPAG9, SRGN, NAPSA, TNFSF9, KLF4, NFKBIE, ILIB, EHDI, PDE4B, PLAUR, EZR, IL1R2, ATP1B3, CRIPI, CD86, MANF, BTG1, TNFAIP2, NDRG1, HSPA5, DUSP5, LYZ, IFRD1, AREG, ARL4C, SI00A11, TRIM25, FN1, REL, ISGI5, S100A4, RUNX3, IL1RN, KDM6B, IFITMI, IGKC, ADORA2B, FCGR2C, FCGR2B, FCGR2A, MDM2, NLRP3, CD44, VEGFA, MXD1, HLA-DMB, SELENOK, NFKBIA, S100A6, CCRL2, NFE2L2, LITAF, CD74, CCR1, CD9, PLAC8, ODC1, CYTIP, MCL1, C15orf48, CD14, SI00A10, ASS1, INHBA, PRDX6, BCL2L11, IER3, TLR2, EMPI, GPR35, ATOX1, SLFN12, HILPDA, SPP1, HIF1A, and NR4A2. This PKCδ KO-like gene score was then applied to a cohort of 60 metastatic melanoma patients treated with anti-PD-1-based immunotherapy, from whom baseline tumor bulk RNAseq data were available. When patients were analyzed with respect to clinical benefit, the inventors found that the group exhibiting disease control had a significantly higher PKCδ KO-like gene signature score compared to patients with progressive disease (FIG. 4G). Together, these results suggest that patients with tumors showing a gene expression profile comparable to that caused by PKCδ-deficiency in myeloid cells have improved response to anti-PD-1 immunotherapy.

### Example 5—Materials and Methods for Aspects Herein

### Melanoma T Cell-Inflamed Gene Signature Score

A list of 13 genes was previously shown to be associated with a T cell-inflamed tumor microenvironment, consisting of CCL2, CCL3, CCL4, CXCL9, CXCL10, CD8A, GZMK, HLA-DMA, HLA-DMB, HLA-DOA, HLA-DOB, ICOS, IRFI³⁰. Using the cancer genome atlas skin cutaneous melanoma dataset (TCGA-SKCM), the inventors fit a sparse linear regression (Elastic Net) by modeling the independent response vector as the mean of the 13 empirically studied genes, and the dependent variables as a matrix of genome-wide gene expression values (around 18,000 transcripts). The model selected 69 genes associated with the 13 gene empirical signature which were as follows: AIM2, ANKRD22, APOL4, B2M, C16orf54, C4orf7, CCL4L2, CCL5, CCL8, CD244, CD38, CD52, CD69, CD74, CD80, CD8B, CD96, CIITA, CRTAM, CST7, CTLA4, CXCL11, CXCR6, DOCK8, FAM26F, FASLG, GBP1, GBP2, GBP4, GBP5, GPR18, GZMA, GZMH, HAPLN3, HAVCR2, HLA-B, HLA-DPAJ, HLA-DPBI, HLA-DPB2, HLA-DQA2, HLA-DQB1, HLA-DRA, HOXAS, IFNG, IGSF6, ILJSRA, IL18BP, ITGAL, KLHL5, LAG3, LGALS2, LY9, PIM1, PLA2G7, PLEK, PSMB9, PTGER4, RASGEFIB, SH2D1A, SLA2, SPN, THBD, TNF, TNIP3, TRAT1, UBD, XCL1, MRP1, ZBED2. This combined list of 82 genes is hereafter referred to as the T cell-inflamed gene signature.

### Genome-Wide Association Study

A total of 258 samples were accessed from TCGA-SKCM. To form the phenotype for a genome wide association study (GWAS), RNAseq values were log 2 normalized, centered, and scaled. For each individual patient, the mean expression level for all 82 genes representing the averaged T cell inflamed gene signature score was computed. The following quality controls were utilized: SNPs were filtered based on having a minor allele frequency (MAF) of >0.10, and a Hardy-Weinberg Equilibrium (HWE) >0.005. Individuals were filtered based on European ancestry, relatedness <0.05, and SNP missingness at <0.05. Bonferroni's method was used to correct for multiple testing to determine genome-wide significance. The inventors subsequently interrogated the genome-wide significant SNP rs1483185, rs1483186 and rs750170 as expression quantitative trait loci (eQTLs) using MatrixEQTL in a lymphoblastoid cell line dataset^{31,32}.

### Calculation of Gene Signature Scores for Expanded TCGA Samples

A quantitative scoring system was used to assign a value to each tumor sample in TCGA based on the expression profile 160 genes from a T cell-inflamed gene signature previously described³³. For the M1/M2 ratio, externally validated M1 and M2 lists generated from the CIBERSORT regression model were utilized²³. RNA sequencing gene expression data for melanoma and lung squamous was downloaded from TCGA data in the form of Z scores from the PanCancerAtlas^{34,35}. Each signature score is the summation of all the Z scores for the genes in that list. The M1/M2 ratio was calculated by dividing the summed Ml score by the summed M2 score, yielding a single number. TCGA cases were labeled as homozygous minor, heterozygous, or homozygous major for each single nucleotide variant (SNV) at the alleles of interest for PRKCD (rs143185, rs143186, and rs1483185). The T cell-inflamed gene signature score and M1/M2 scores were then compared between each germline SNV group by ordinary one-way ANOVA and multiple comparisons (GraphPad, Prism 9).

### Generation of Mouse Bone Marrow Chimeras

PKCδ-KO (Prkcd^{-/-}) mice were obtained as a generous gift from the lab of Dr. C.

Ronald Kahn and bred at the University of Chicago¹¹. Prkcd^{+/+} (WT) or Prkcd^{-/-} (KO) donor mice were sacrificed at 6-10 weeks. Femurs and tibias were collected and flushed with PBS using a 26G needle. Kneecaps were minced using scissors and the bone marrow was passed through a 70 uM filter (Fisher, 352350). CD90.2 cells were then depleted via MACS column separation (Miltenyl, 130-121-278) before bone marrow cells were counted and resuspended in PBS for sterile intravenous injection. 24 hours before engraftment, B6.SJL-Ptprc^{α}/BoyAiTac mice (Taconic, 4007, CD45.1) sex-matched transplant recipient mice were irradiated with 5 Gy and 5.5 Gy (Cesium 137 source) with a 3-hour rest period in between. Bone marrow was injected intravenously via retro-orbital injection with a minimum of 4.0×10⁶ cells injected per host mouse in a volume of 100ul PBS. Mice were allowed to rest of a minimum of 8 weeks before experimental use. All animal procedures were approved by the Institutional Animal Care and Use Committee of the University of Chicago.

### Tumor Implantation

B16-F10-SIY-dsRed (referred to as B16-SIY subsequently) were thawed, grown in cDMEM media (DMVEM, 10% FBS, 1% NEAA, 1% Penn-Strep, 1% MOPS buffer) and passaged 2-5 times before experimentation. Cells were washed 3× with PBS before being resuspended at 2×10⁶ cells per 100ul of PBS and 100ul of cells was injected subcutaneously into the right flank. The length, width, and height of a tumor was measured by calipers 3 times a week and the volume was calculated using the following formula. (4/3*pi*L/2*W/2*H/2)

$$\frac{4}{3} \times π \times \frac{L}{2} \times \frac{W}{2} \times \frac{H}{2} \frac{4}{3} \times π \times \frac{L}{2} \times \frac{W}{2} \times \frac{H}{2}$$

### Cell Isolation

Tumors were excised, mechanically dissociated, and incubated with a mixture of 10 mg/ml collagenase type IV (Sigma, C5138), 1 mg/ml hydrogenase type V (Sigma, H6254), and 2,000 units/ml DNAse type IV (Sigma, D5025-150ku) for 45 minutes at 37C° before being washed with PBS through a 70 uM filter (Corning, 352350). Tumors were then passed through a Ficoll-Paque gradient (Cytivia Life Sciences, 17144003), and the bilayer was collected. Spleens were mechanically dissociated and washed through a 70 uM filter before RBC lysis by incubation with Gey's solution for 2 minutes at room temperature.

### Flow Cytometry and Cell Sorting

Cells were incubated with appropriate antibodies (Table 3) in FACS buffer (PBS, 5% FBS, 10 uM EDTA) for at least 30 minutes at 4C°. When necessary, cells were fixed in 1% PFA or fixed and permeabilized using a kit (Invitrogen, 00-5523-00). Any intracellular staining was performed overnight at 4C°. Both traditional (BD Fortessa 4-15, BD LSR Fortessa X-20) and spectral flow cytometers (Cytek Aurora) were employed, and analysis performed using FlowJo software (Treestar). Flouresence activated cell sorting (FACS) was performed entirely under sterile conditions. Sorting was performed on a AriaIIIu (BD) and samples were sorted into FACS buffer before further analysis. To ensure purity, post-sort samples were run immediately after initial sort set up, and a given population of interest was required to be >90% of all live cells. Percentages of T cells was calculated as follows ((Number of living cells in population/Number of living CD3⁺ T cells)X 100). Number per gram tumor was calculated as follows ((number of living cells/tumor weight)X sample dilution factor). A list of all antibodies can be found in Table 3.

### Table 3

TABLE 3

| Antigen | Fluorophore | clone |
|---|---|---|
| Caspase3 | BV605 | C92-605 |
| CD70 | BV711 | FR70 |
| Ki-67 | BV711 | B56 |
| Brilliant Stain Buffer | N/A | N/A |
| Brilliant Stain Buffer Plus | N/A | N/A |
| CD69 | BUV563 | H12F3 |
| CD72 | BUV395 | K10.6 |
| CD86 | BUV496 | PO3 |
| CD8a | BUV395 | 53-6.7 |
| Ly6G | BUV661 | 1A8 |
| CD11c | AF647 | N418 |
| CD4 | PerCP-Cy5.5 | RM4-5 |
| FC BLOCK | None | 93 |
| CD103 | BV421 | 2E7 |
| CD11b | APC | M1/70 |
| CD11b | BV570 | M1/70 |
| CD11c | BV650 | N418 |
| CD14 | FITC | Sa14-2 |
| CD19 | AmCyan BV510 | 6D5 |
| CD206 | BV605 | C068C2 |
| CD3 | (PacBlue) BV421 | 17A2 |
| CD3 | Alexa Fluor 700 | 17A2 |
| CD3 | APC-Cy7 | 145-2C11 |
| CD3 | BV711 | 17A2 |
| CD4 | BV605 | RM4-5 |
| CD4 | BV711 | RM4-5 |
| CD4 | Spark NIR 685 | GK1.5 |
| CD45.1 | Pac Blue | A20 |
| CD45.2 | APC-Cy7 | 104 |
| CD45.2 | BV785 | 104 |
| CD80 | PE/Dazzle 594 | 16-10A1 |
| CD84 | PE | mCD84.7 |
| CD8a | BV510 | 53-6.7 |
| CD8a | BV605 | 53-6.7 |
| CD8a | BV711 | 53-6.7 |
| CXCR2 (CD182) | PE-Cy7 | SA044G4 |
| F4/80 | FITC | BM8 |
| I-A/I-E | Pacific Blue | M5/114.15.2 |
| I-A/I-E | PE-Cy7 | M5/114.15.2 |
| Fixable Viability | Zombie NIR | |
| Ly6C | Alexa Fluor 700 | HK1.4 |
| Ly6C | BV785 | HK1.4 |
| NK1.1 | APC-Cy7 | PK136 |
| CD8a | PerCP-eFluor 710 | 53-6.7 |
| F4/80 | PerCP-Cy5.5 | BM8 |
| Foxp3 | Alexa Fluor 488 | FJK-16s |
| Fixable Viability | eFluor506 | |
| CD45 | AF532 | 30-F11 |
| SIY | PE | SIYRYYGL |

### Enzyme-Linked Immunospot (ELISPOT) Assay

Spleens from tumor-naive WT-engrafted mice, tumor-bearing WT-engrafted mice, or tumor-bearing KO-engrafted mice were collected, with each individual mouse being considered an experimental replicate. Splenic cells were isolated as described above and plated at 1×10⁶ cells per well in T cell growth media (DMEM, 10% FB S, 10% NEAA, 10% Penn-Strep, 10% MOPS buffer, 0.1 mM beta-2ME). Negative control wells received T cell growth media only, while experimental wells received 160 nM of H-2K^{b} SIY peptide (SIYRYYGL), and positive control wells received PMA (50 ng/ml) and Jonomycin (500 ng/ml). Each experimental replicate had the following technical replicates which were then averaged for each group: 2 negative control wells, 3 SIY peptide wells, and 1 positive control well. ELISPOT was performed according to the manufacture's protocol using the BD Mouse IFN-γ Elispot kit (BD Biosciences, 551083).

### Anti-PD-L 1 Therapy

In bone marrow-engrafted animals, B16-SIY tumors were established until an average volume of 100 mm³ was reached and then mice were split into control or treatment groups. The average volume of each group was matched at the time of this separation. 3 times per week, 100ug of anti-PD-L1 (B7-H1, BioXcell, BE0101) was administered in 100ul of PBS via intraperitoneal (i.p.) injection, while control mice received 100ul of PBS via i.p. injection on the same days. In conditional KO animals, 100ug of anti-PD-L1 (B7-H1, BioXcell, BE0101) was administered in 100ul of PBS via i.p. injection 3 times per week starting 7 days after tumor implantation for 5 total doses, with control mice receiving 100ul of PBS via i.p. injection on the same days.

### Single Cell RNA Sequencing

Engrafted mice were injected with B16-SIY and sacrificed after 26 days on 2 separate occasions and processed independently. On each collection day, immune cells from tumors were isolated as described above and stained with Live-Dead (APC-Cy7) and CD45.2 (FITC). Live CD45.2⁺ cells were sorted into FACS buffer before being washed 3× in PBS. Single cell partitioning and library creation was performed by the University of Chicago Genomics core using a chromium controller from 10× genomics following the “Chromium Single Cell 3′ Reagent Kits v3” user guide and associated reagents. 10,000 cells were targeted and encapsulated per sample. Libraries were sequenced in PE100 format of an SP-100 flowcell of an Illumina Novaseq 6000 instrument.

Processing and Analysis of scRNAseq Data

Demultiplexing and alignment to the mm10 transcriptome (version 3.0.0) was performed, with count matrices created using the Cell Ranger pipeline (version 3.0.2). scRNA-seq count matrices were read by Seurat (version 3.2.3)³⁶, and a single Seurat object was created from all samples from both collection days. Quality control filtering of cells was based on the following: less then 5% mitochondria genes expressed, with a minimum of 200 counts, and the number of unique molecular identifiers (UMIs) was set between 50 and 3,000. Overall, 41,187 cells passed the quality control metrics. Datasets were normalized using the “SCTransform” function and the top 3,000 features were identified using “SelectIntegrationFeatures”. These were used as anchor features while the Seurat object was prepared for integration using “PrepSCTIntegration”, then all datasets were integrated using the “FindIntegrationAnchors” function. The scaled and integrated data was then processed using “RunPCA” with the first 100 principal components, followed by UMAP clustering analysis. Optimal UMAP resolution was determined by examining each resolution between 0.1 to 0.9 using 0.1 steps using the clustree r package³⁷. A final resolution of 0.6 was chosen and markers for each cluster were determined using the “FindAllMarkers” function with a min.pct of 0.25 and a log fold change threshold of 0.25. In total 24 unique clusters were identified, and each cluster was labeled by identifying a top uniquely expressed genes when compared to all other clusters. For each unique cluster, “FindMarkers” was used to identify differentially expressed genes between WT and KO samples using the MAST test method. Genes that had an fdr >=0.05 and a log fold change magnitude >=0.25 were considered significant. The fgsea package was used to perform gene set enrichment analysis for all DEGs in each cluster. The DoHeatmap package was used to illustrate differences in select genes by plotting the gene expression log-averaged across all cells, in a given cluster for each biological replicate.

### Conditional KO Mouse Generation

APrkcd^{fl/fl} conditional KO line was generated using Cyagen's TurboKnockout® gene targeting service. Exons 7-9 were targeted for conditional KO in C57BL/6J ES cells (FIG. 8). Deletion of this region predicted a complete loss of function for the protein and was confirmed by genotyping using the primers of Table 4:

TABLE 4

| Target | Primer 1 | Primer 2 |
|---|---|---|
| PKCd | GCTCTATTGCCTCGGCTTCAT (SEQ | AGGTGAGAAAGACAGCAAAGGG |
| | ID NO. 1) | (SEQ ID NO. 2) |
| LacZ | TGATGCGGTGCTGATTACGAC | GTCAAAACAGGCGGCAGTAAG |
| | (SEQ ID NO. 3) | (SEQ ID NO. 4) |
| PKCdcKO | TATGTTTTGAGAGCTTAGCAGGTG | GATGCTGTATAGGGGCAGGAAAG |
| Target | (SEQ ID NO. 5) | (SEQ ID NO. 6) |
| PKCdcKO | TATGTTTTGAGAGCTTAGCAGGTG | GATGCTGTATAGGGGCAGGAAAG |
| Del | (SEQ ID NO. 7) | (SEQ ID NO. 8) |
| Vav1- | AGATGCCAGGACATCAGGAACCT | ATCAGCCACACCAGACACAGAGAT |
| iCre | G ((SEQ ID NO. 9) | C (SEQ ID NO. 10) |
| LysM- | CCCAGAAATGCCAGATTACG (SEQ | CTTGGGCTGCCAGAATTTCTC (SEQ |
| Cre | ID NO. 11) | ID NO. 12) and/or |
| | | TTACAGTCGGCCAGGCTGAC (SEQ |
| | | ID NO. 13) |

Two independent strains were developed, labeled 2B4 and 2C1. The main strain featured throughout the paper is 2B4 although no functional differences were observed at any point between the strains. Both were crossed with B6.Cg-Commd10^{Tg(Vavl-iCre)A2Kio}/J (Jackson, 008610, referred to subsequently as “Vavl-iCre”) as well as B6.129P2-Lyz2^{tml(Cre)Ifo}/J (Jackson, 004781, referred to subsequently as “LysM-Cre”) at the University of Chicago. Female littermates between the ages of 7 and 12 weeks were used for all experiments.

RNA isolation and qRT-PCR

Immune cells from tumors are isolated and sorted as described above. Tumor associated macrophages (TAMs) marked as Live-Dead-CD45⁺ CD90.2⁻ B220⁻ CD11b⁺ Ly6G⁻ F4/80⁺ were isolated before being washed 3×in cold PBS. RNA was isolated using Qiagen RNeasy micro kit (Qiagen 74004) and cDNA was created using the High-Capacity cDNA Reverse Transcription Kit with RNase Inhibitor (Applied Biosystems™, 4374966). qPCR reactions were carried out using TaqMan master mix (Life Technologies) and defined primer/probe sets (Roche universal probe library). Reactions primers for Trem2, Illb, and TNF genes were selected using the Roche primer design center website (discontinued) and the Roche universal probe library was used. Reactions were run on a StepOnePlus™ Real-Time PCR System (Applied Biosystems, 4376600) in MicroAmp™ Fast Optical 96-Well reaction plates (Applied Biosystems, 434907). Each sample was run in duplicate wells and the CT of those wells was averaged before expression levels were calculated as follows ΔCT=CT_{Gapdh}−CT_{Gene of interest}; expression level=2^{ΔCT}.

### Melanoma Patient Bulk RNAseq Analysis

Patient data was collected according to the Biological Sciences Division Institutional Review Boards at the University of Chicago under the protocols 15-0837, 15-0788, and 17-0686. Samples were either formalin-fixed paraffin-embedded or fresh frozen before they were submitted for bulk RNA sequencing at the University of Chicago sequencing core. Patients were selected as follows. Metastatic melanoma patients that received immunotherapy after sample collection and had no history of prior immunotherapy treatments. Patients with Uveal, Acral, Mucosal types were removed, leaving cutaneous and unknown primary types. All tumor samples were collected before treatment anti-PD-1 based therapy. Patients were required to have clinical outcome data marked as either progressive disease (PD), stable disease (SD), partial response (PR), or complete response (CR). Kallisto and tximport were used to align to gencode primary assembly sequence (release 39) and export sequencing data while Limma and edgeR R packages were used to process the count matrices. Samples were filtered by a minimum of 5,100,000 reads and genes were filtered using the following cutoffs: a minimum of 30 samples were required to express a given gene and with a minimum counts per million (CPM) of 1. Normalization was done using the “calcNormFactors” function, using the TMM method. Count data was transformed using “limmavoom” and data was centered using the scale function in R. The PKC6-KO-related gene signature was created from genes in the Clqa cluster that were increased in KO animals and had a positive fold change >0.25 and a fdr<0.05. The biomaRt R package was used to convert the mouse gene list into their human orthologs. Any genes that did not have a match were dropped. 119 of 129 total genes were identified to have human counterparts and utilized for the signature. The median expression of all signature genes was calculated for each patient and then plotted via violin plot. Patients were grouped by either “progression” which consisted of the progressive disease (PD) group, or “disease control” which consisted of stable disease (SD), partial response (PR), and complete response (CR). A student's t-test was performed on the data to assess significance.

### Statistical Analysis

All statistical analyses were performed using GraphPad Prism (Graphpad) with the exception of the GWAS and scRNAseq analysis. Unless otherwise noted, all data are shown as mean±s.e.m. and significance assumed with P≤0.05. Significance for growth curves was determined by two-way ANOVA and Sidak's Multiple Comparisons test. Significance for bar graphs determined by unpaired t test. Significance for data presented in violin plots was determined by ordinary one-way ANOVA and Tukey's multiple comparisons test. Survival curves were determined by Kaplan-Meier survival analysis and p values for Gehan-Brewslow-Wilcoxon test were reported.

### Example 6—PKCd Germline Variants and Genetic Deletion in Mice Augment Anti-Tumor Immunity Through Regulation of Myeloid Cells

Certain aspects validate the approach of using germline susceptibility loci to identify novel targets involved in tumor immunity. The data add to recent literature describing associations between selected autoimmune-related SNPs and checkpoint blockade efficacy²⁴,2. The genome-wide approach herein identified PRKCD as a novel candidate, suggesting that decreased expression might lead to augmented anti-tumor immunity. Using gene-targeted mice, the inventors found that hematopoietic loss of PKCd led to improved immune-mediated tumor growth control, which synergized with anti-PD-L1 therapy and was associated with a shift towards inflammatory gene expression in TAMs. It has been described that TAMs trend towards M2-like programming, and that a relatively high ML.M2 ratio is associated with improved clinical outcomes across multiple tumor types²⁶-2⁸. Certain aspects revealed that PKCd loss caused altered expression of multiple genes indicative of a shift towards the M1 differentiation state. Among specific genes, Trem2 has been reported to be overrepresented in the macrophages of melanoma patients that failed to respond to immune checkpoint therapy²⁰, which also showed decreased expression in PKCd KO TAMs. Conversely, common inflammatory markers like CD14 were highly expressed in PKCd KO TAMs. Consistent with these results, a recent CRISPR screen revealed that loss of PKCd led to increased CD14 expression in myeloid cells²⁹. Certain aspects showing that genetic loss of PKCd selectively in the LysM⁺ compartment was sufficient for tumor growth delay and improved response to anti-PD-L1 therapy support a central functional role for PKCd within the myeloid compartment. Inasmuch as therapeutic strategies aiming to decrease M2 phenotype macrophages from the tumor microenvironment and promote an M1 state are highly desirable yet still lacking, certain aspects suggest that PKCd may be an attractive therapeutic target in this regard. The biochemical mechanisms by which PKCd influences the M1/M2 differentiation state are not yet clear and an important subject for future investigation. The fact that CRISPR-mediated deletion was reported to increase CD14 gene expression argues that PKCd is involved in a signaling pathway promoting an M2-state while restricting MI-state genes. As a kinase, understanding the upstream ligands that trigger PKCd activation, as well as the downstream phosphorylation substrates of this molecule specifically in myeloid cells, will ultimately be insightful. These unknowns notwithstanding, pursuing allosteric inhibitors that preferentially inhibit PKCd activity while sparing other important PKC isoforms should be prioritized for future development.

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, and those cited elsewhere herein, to the extent that they provide exemplary procedural or other details supplementary to those set forth herein, are specifically incorporated herein by reference.
Chat V, Ferguson R, Simpson D, et al. Autoimmune genetic risk variants as germline biomarkers of response to melanoma immune-checkpoint inhibition. Cancer Immunol Immunother. 2019; 68(6):897-905. doi:10.1007/s00262-019-02318-8
Kikkawa U, Matsuzaki H, Yamamoto T. Protein kinase C delta (PKC delta): activation mechanisms and functions. J Biochem. 2002; 132(6):831-839. doi:10.1093/oxfordjournals.jbchem.a003294
Oshi M, Tokumaru Y, Asaoka M, et al. M1 Macrophage and M1/M2 ratio defined by transcriptomic signatures resemble only part of their conventional clinical characteristics in breast cancer. Sci Rep. 2020; 10(1):16554. Published 2020 Oct 6. doi:10.1038/s41598-020-73624-w
1. Ayers, M. et al. IFN-γ-related mRNA profile predicts clinical response to PD-1 blockade. J. Clin. Invest. 127, (2017).
2. Tumeh, P. C. et al. PD-1 blockade induces responses by inhibiting adaptive immune resistance. Nature 515, 568-571 (2014).
3. Danaher, P. et al. Pan-cancer adaptive immune resistance as defined by the Tumor Inflammation Signature (TIS): Results from The Cancer Genome Atlas (TCGA). J. Immunother. Cancer 6, (2018).
4. Gokuladhas, S., Schierding, W., Golovina, E., Fadason, T. & O'Sullivan, J. Unravelling the Shared Genetic Mechanisms Underlying 18 Autoimmune Diseases Using a Systems Approach. Front. Immunol. 12, 3262 (2021).
5. Spranger, S. & Gajewski, T. Rational combinations of immunotherapeutics that target discrete pathways. J. Immunother. Cancer 1, 1 (2013).
6. Stranger, B. E. et al. Population genomics of human gene expression. Nat. Genet. 39, 1217-1224 (2007).
7. Spranger, S. et al. Density of immunogenic antigens does not explain the presence or absence of the T-cell-inflamed tumor microenvironment in melanoma. Proc. Natl. Acad. Sci. 113, E7759-E7768 (2016).
8. Kuehn, H. S. et al. Loss-of-function of the protein kinase C d (PKCd ) causes a B-cell lymphoproliferative syndrome in humans. Blood 121, 3117-3126 (2013).
9. Belot, A. et al. Protein kinase C6 deficiency causes mendelian systemic lupus erythematosus with B cell-defective apoptosis and hyperproliferation. Arthritis Rheum. 65, 2161-2171 (2013).
10. Miyamoto, A. et al. Increased proliferation of B cells and auto-immunity in mice lacking protein kinase C6. Nature 416, 865-869 (2002).
11. Bezy, O. et al. PKCδ regulates hepatic insulin sensitivity and hepatosteatosis in mice and humans. J. Clin. Invest. 121, 2504-2517 (2011).
12. Ma, W., Baumann, C. & Viveiros, M. M. Lack of protein kinase C-delta (PKCS) disrupts fertilization and embryonic development. Mol. Reprod. Dev. 82, 797-808 (2015).
13. Heng, T. S. P. et al. The Immunological Genome Project: networks of gene expression in immune cells. Nat. Immunol. 2008 910 9, 1091-1094 (2008).
14. Fujimura, T., Kambayashi, Y., Fujisawa, Y., Hidaka, T. & Aiba, S. Tumor-associated macrophages: Therapeutic targets for skin cancer. Front. Oncol. 8, 3 (2018).
15. Li, C. et al. Tumor-associated macrophages: potential therapeutic strategies and future prospects in cancer. J. Immunother. Cancer 9, e001341 (2021).
16. Stout, R. D. et al. Macrophages Sequentially Change Their Functional Phenotype in Response to Changes in Microenvironmental Influences. J. Immunol. 175, 342-349 (2005).
17. Zanoni, I. & Granucci, F. Role of CD14 in host protection against infections and in metabolism regulation. Front. Cell. Infect. Microbiol. 4, 32 (2013).
18. Molgora, M. et al. TREM2 Modulation Remodels the Tumor Myeloid Landscape Enhancing Anti-PD-1 Immunotherapy. Cell 182, 886-900.e17 (2020).
19. Arlauckas, S. P. et al. Arg1 expression defines immunosuppressive subsets of tumor-associated macrophages. Theranostics 8, 5842 (2018).
20. Xiong, D., Wang, Y. & You, M. A gene expression signature of TREM2hi macrophages and 76 T cells predicts immunotherapy response. Nat. Commun. 2020 111 11, 1-12 (2020).
21. Dobrovolskaia, M. A. & Vogel, S. N. Toll receptors, CD14, and macrophage activation and deactivation by LPS. Microbes Infect. 4, 903-914 (2002).
22. Marzio, R., Jirillo, E., Ransijn, A., Mau6l, J. & Corradin, S. B. Expression and function of the early activation antigen CD69 in murine macrophages. J. Leukoc. Biol. 62, 349-355 (1997).
23. Newman, A. M. et al. Robust enumeration of cell subsets from tissue expression profiles. Nat. Methods 12, 453-457 (2015).
24. Chat, V. et al. Autoimmune genetic risk variants as germline biomarkers of response to melanoma immune-checkpoint inhibition. Cancer Immunol. Immunother. 68, 897-905 (2019).
25. Breunis, W. B. et al. Influence of cytotoxic T lymphocyte-associated antigen 4 (CTLA4) common polymorphisms on outcome in treatment of melanoma patients with CTLA-4 blockade. J. Immunother. 31, 586-590 (2008).
26. Jayasingam, S. D. et al. Evaluating the Polarization of Tumor-Associated Macrophages Into M1 and M2 Phenotypes in Human Cancer Tissue: Technicalities and Challenges in Routine Clinical Practice. Front. Oncol. 9, 1512 (2020).
27. Kakizaki, A. et al. Immunomodulatory effect of peritumorally administered interferon-beta on melanoma through tumor-associated macrophages. Oncoimmunology 4, (2015).
28. Pathria, P., Louis, T. L. & Varner, J. A. Targeting Tumor-Associated Macrophages in Cancer. (2019). doi:10.1016/j.it.2019.02.003
29. Jimenez-Duran, G. et al. Pharmacological validation of targets regulating CD14 during macrophage differentiation. EBioMedicine 61, (2020).
30. Spranger, S., Bao, R. & Gajewski, T. F. Melanoma-intrinsic 0-catenin signalling prevents anti-tumour immunity. Nature 523, 231-235 (2015).
31. Shabalin, A. A. Matrix eQTL: ultra fast eQTL analysis via large matrix operations. Bioinformatics 28, 1353-1358 (2012).
32. Lappalainen, T. et al. Transcriptome and genome sequencing uncovers functional variation in humans. Nature 501, 506-511 (2013).
33. Luke, J. J., Bao, R., Sweis, R. F., Spranger, S. & Gajewski, T. F. WNT/b-catenin pathway activation correlates with immune exclusion across human cancers. Clin. Cancer Res. 25, 3074-3083 (2019).
34. Hoadley, K. A. et al. Cell-of-Origin Patterns Dominate the Molecular Classification of 10,000 Tumors from 33 Types of Cancer. Cell 173, 291-304.e6 (2018).
35. Gao, J. et al. Integrative analysis of complex cancer genomics and clinical profiles using the cBioPortal. Sci. Signal. 6, 1-2 (2013).
36. Stuart, T. et al. Comprehensive Integration of Single-Cell Data. Cell 177, 1888-1902.e21 (2019).
37. Zappia, L. & Oshlack, A. Clustering trees: a visualization for evaluating clusterings at multiple resolutions. Gigascience 7, 1-9 (2018).

1. A method of treating a subject for cancer comprising administering to the subject a therapeutically effective amount of a PKC-delta inhibitor and a checkpoint blockade therapy, wherein the subject was determined to be resistant to a previous treatment, and wherein the PKC-delta inhibitor comprises a nucleic acid, rottlerin, a rottlerin analog, a rottlerin derivative, BJE6-106, and/or delcasertib.

2. A method of treating a subject for cancer comprising administering to the subject a therapeutically effective amount of a PKC-delta inhibitor and an immunotherapy.

3. The method of claim 2, wherein the immunotherapy is a checkpoint blockade therapy.

4. The method of claim 3, wherein the checkpoint blockade therapy comprises an anti-PD1 antibody, anti-PD-L1 antibody, or anti-CTLA4 antibody.

5. The method of claim 2, wherein the immunotherapy is an adoptive immune cell therapy.

6. The method of claim 2, wherein the immunotherapy is a CAR immune cell therapy.

7. The method of any of claims 2-6, wherein the PKC-delta inhibitor is a nucleic acid.

8. The method of claim 7, wherein the PKC-delta inhibitor is a small interfering RNA, a small hairpin RNA, or an antisense oligonucleotide.

9. The method of claim 7, wherein the PKC-delta inhibitor is rottlerin or an analog or derivative thereof.

10. The method of claim 7, wherein the PKC-delta inhibitor is BJE6-106.

11. The method of claim 7, wherein the PKC-delta inhibitor is delcasertib.

12. The method of any of claims 2-11, wherein the PKC-delta inhibitor is linked to a myeloid cell targeting agent.

13. The method of claim 12, wherein the myeloid cell targeting agent is an antibody that specifically binds to a myeloid cell surface protein.

14. The method of claim 13, wherein the myeloid cell surface protein is CD14, CD86, CD69, or CD16.

15. The method of any of claims 2-14, wherein the immunotherapy and the PKC-delta inhibitor are administered simultaneously.

16. The method of any of claims 2-14, wherein the immunotherapy and the PKC-delta inhibitor are administered sequentially.

17. The method of claim 16, wherein the immunotherapy is administered prior to administering the PKC-delta inhibitor.

18. The method of claim 16, wherein the immunotherapy is administered subsequent to administering the PKC-delta inhibitor.

19. The method of any of claims 2-18, wherein the subject has melanoma.

20. The method of any of claims 2-18, wherein the subject has lung cancer.

21. The method of any of claims 2-20, wherein the subject does not have a RAS mutant cancer.

22. The method of any of claims 2-21, wherein the subject was previously treated for cancer.

23. The method of claim 22, wherein the subject was determined to be resistant to the previous treatment.

24. A method of treating a subject for cancer comprising administering to the subject a therapeutically effective amount of a PKC-delta inhibitor linked to a myeloid cell targeting agent.

25. The method of claim 24, wherein the myeloid cell targeting agent is an antibody that specifically binds to a myeloid cell surface protein.

26. The method of claim 25, wherein the myeloid cell surface protein is CD14, CD86, or CD16.

27. The method of any of claims 24-26, wherein the PKC-delta inhibitor is a nucleic acid.

28. The method of claim 27, wherein the PKC-delta inhibitor is a small interfering RNA, a small hairpin RNA, or an antisense oligonucleotide.

29. The method of any of claims 24-26, wherein the PKC-delta inhibitor is rottlerin or an analog or derivative thereof.

30. The method of any of claims 24-26, wherein the PKC-delta inhibitor is BJE6-106.

31. The method of any of claims 24-26, wherein the PKC-delta inhibitor is delcasertib.

32. The method of any of claims 24-31, further comprising administering to the subject an immunotherapy.

33. The method of claim 32, wherein the immunotherapy is a checkpoint blockade therapy.

34. The method of claim 33, wherein the checkpoint blockade therapy comprises an anti-PD1 antibody, anti-PD-L1 antibody, or anti-CTLA4 antibody.

35. A method of treating a subject for cancer comprising:
(a) subjecting immune cells ex vivo to conditions sufficient to reduce PKC-delta expression in the immune cells; and
(b) subsequent to (a), administering the immune cells to the subject.

36. The method of claim 35, wherein (a) comprises deleting at least a portion of the PRKCD gene in the immune cells.

37. The method of claim 35 or 36, wherein the immune cells comprise myeloid cells.

38. The method of any of claims 35-37, further comprising administering to the subject an additional immunotherapy.

39. The method of claim 38, wherein the additional immunotherapy is a checkpoint blockade therapy.

40. The method of claim 39, wherein the checkpoint blockade therapy comprises an anti-PD1 antibody, anti-PD-L1 antibody, or anti-CTLA4 antibody.

41. A method of treating a subject for cancer comprising administering to the subject an effective amount of immune cells that do not express PKC-delta.

42. The method of claim 41, wherein the immune cells comprise a deletion in the PRKCD gene.

43. The method of claim 41 or 42, wherein the immune cells comprise myeloid cells.

44. The method of any of claims 41-43, wherein the immune cells comprise CD8^{m} T cells.

45. The method of any of claims 41-44, further comprising administering to the subject an additional immunotherapy.

46. The method of claim 45, wherein the additional immunotherapy is a checkpoint blockade therapy.

47. The method of claim 46, wherein the checkpoint blockade therapy comprises an anti-PD1 antibody, anti-PD-L1 antibody, or anti-CTLA4 antibody.

48. A method of treating a subject for cancer comprising administering an immunotherapy to a subject determined to have decreased PKC-delta expression relative to a control or reference subject.

49. The method of claim 38, wherein the subject was determined to have decreased PKC-delta expression in hematopoietic cells from the subject.

50. The method of claim 49, wherein the hematopoietic cells are myeloid cells.

51. The method of any of claims 48-50, wherein the subject was further determined to have a single nucleotide polymorphism (SNP) in the PRKCD gene.

52. The method of claim 51, wherein the SNP is rs1483185, rs1483186, or rs750170.

53. The method of any of claims 48-52, wherein the immunotherapy is a checkpoint blockade therapy.

54. The method of claim 53, wherein the checkpoint blockade therapy comprises an anti-PD1 antibody, anti-PD-L1 antibody, or anti-CTLA4 antibody.

55. A method of treating a subject for cancer comprising administering an immunotherapy to a subject determined to have a SNP in the PRKCD gene, wherein the SNP is rs1483185, rs1483186, orrs750170.

56. The method of claim 55, wherein the subject was further determined to have decreased PKC-delta expression relative to a control or reference subject.

57. The method of claim 56, wherein the subject was determined to have decreased PKC-delta expression in hematopoietic cells from the subject.

58. The method of any of claims 55-57, wherein the hematopoietic cells are myeloid cells.

59. The method of any of claims 55-58, wherein the immunotherapy is a checkpoint blockade therapy.

60. The method of claim 59, wherein the checkpoint blockade therapy comprises an anti-PD1 antibody, anti-PD-L1 antibody, or anti-CTLA4 antibody.

61. A method of treating a subject for a RAS wild-type cancer comprising administering to the subject an effective amount of a PKC-delta inhibitor.

62. The method of claim 61, wherein the PKC-delta inhibitor is a nucleic acid.

63. The method of claim 62, wherein the PKC-delta inhibitor is a small interfering RNA, a small hairpin RNA, or an antisense oligonucleotide.

64. The method of claim 62, wherein the PKC-delta inhibitor is rottlerin or an analog or derivative thereof.

65. The method of claim 62, wherein the PKC-delta inhibitor is BJE6-106.

66. The method of claim 62, wherein the PKC-delta inhibitor is delcasertib.

67. The method of any of claims 61-66, wherein the PKC-delta inhibitor is linked to a myeloid cell targeting agent.

68. The method of claim 67, wherein the myeloid cell targeting agent is an antibody that specifically binds to a myeloid cell surface protein.

69. The method of claim 68, wherein the myeloid cell surface protein is CD14, CD86, or CD16.

70. The method of any of claims 61-68, further comprising administering an immunotherapy to the subject.

71. The method of claim 70, wherein the immunotherapy is a checkpoint blockade therapy.


SEQUENCE LISTING (13 sequences)

SEQ ID NO: 1 - DNA, 21 bp, synthetic construct
  source 1..21: mol_type = other DNA
gctctattgcctcggcttcat

SEQ ID NO: 2 - DNA, 22 bp, synthetic construct
  source 1..22: mol_type = other DNA
aggtgagaaagacagcaaaggg

SEQ ID NO: 3 - DNA, 21 bp, synthetic construct
  source 1..21: mol_type = other DNA
tgatgcggtgctgattacgac

SEQ ID NO: 4 - DNA, 21 bp, synthetic construct
  source 1..21: mol_type = other DNA
gtcaaaacaggcggcagtaag

SEQ ID NO: 5 - DNA, 24 bp, synthetic construct
  source 1..24: mol_type = other DNA
tatgttttgagagcttagcaggtg

SEQ ID NO: 6 - DNA, 23 bp, synthetic construct
  source 1..23: mol_type = other DNA
gatgctgtataggggcaggaaag

SEQ ID NO: 7 - DNA, 24 bp, synthetic construct
  source 1..24: mol_type = other DNA
tatgttttgagagcttagcaggtg

SEQ ID NO: 8 - DNA, 23 bp, synthetic construct
  source 1..23: mol_type = other DNA
gatgctgtataggggcaggaaag

SEQ ID NO: 9 - DNA, 24 bp, synthetic construct
  source 1..24: mol_type = other DNA
agatgccaggacatcaggaacctg

SEQ ID NO: 10 - DNA, 25 bp, synthetic construct
  source 1..25: mol_type = other DNA
atcagccacaccagacacagagatc

SEQ ID NO: 11 - DNA, 20 bp, synthetic construct
  source 1..20: mol_type = other DNA
cccagaaatgccagattacg

SEQ ID NO: 12 - DNA, 21 bp, synthetic construct
  source 1..21: mol_type = other DNA
cttgggctgccagaatttctc

SEQ ID NO: 13 - DNA, 20 bp, synthetic construct
  source 1..20: mol_type = other DNA
ttacagtcggccaggctgac